(19) United States
(12) Patent Application Publication
(10) Pub. No.: US 2009/0306350 A1
(43) Pub. Date: Dec. 10, 2009

(54) NEW PURIFICATION METHOD OF LACTOFERRIN

(75) Inventors: Gerald Rowe, St-laurent (CA); Hafida Aomari, St-laurent (CA); Denis Petitclerc, Lennoxville (CA)

Correspondence Address: DLA PIPER LLP (US);ATTN: PATENT GROUP, P.O. Box 2758, Reston, VA, 20195, US

(73) Assignee: CREA BIOPHARMA INC., Fleurimont, PQ (CA)

(21) Appl. No.: 11/914,357

(22) PCT Filed: May 12, 2006
(86) PCT No.: PCT/CA06/00780

§ 371 Date: Aug. 21, 2009

Related U.S. Application Data
(60) Provisional application No. 60/680,487, filed on May 13, 2005.

(51) Int. Cl. C07K 14/79; C07K 1/16

(52) U.S. Cl. 530/400; 530/417

(57) ABSTRACT

The invention relates to methods for purifying lactoferrin, stabilizing it in solution and improving its activity. In one embodiment of the present invention, it is provided methods for lactoferrin purification employing hydrophobic and/or hydrophilic adsorbent under specific conditions for maintaining or preserving lactoferrin protein stability. It is also provided a process to remove inhibitor of lactoferrin activity.

### FIELD OF THE INVENTION

The invention relates to methods for purifying lactoferrin, stabilizing it in solution and improving its activity.

### BACKGROUND OF THE INVENTION

Lactoferrin (LF) is a single chain, metal-binding glycoprotein of the transferrin family and is part of the innate host defense system played by neutrophils, mucosal surfaces and milk secretion (Lönnerdal and Iyer, Annual Review of Nutrition 15:93-110, 1995). It is capable of binding two molecules of iron per molecule of protein. In vitro, LF has antibacterial (Arnold et al., Science 197:263-265, 1977; Ellison III and Giehl, Clin. Invest. 88:1080-1091, 1991), antifungal (Soukka et al., Fems Microbiol. Lett. 69:223-228, 1992), anti-endotoxin (Zhang et al., Infect. Immun. 67:1353-1358, 1999) and antiviral (Hasegawa et al., Jpn. J. Med. Sci Biol. 47:73-85, 1994; Harmsen et al., J. Infect. Dis 172:380-388, 1995) activities. In fact, Harmsen et al. (1995) stated that “Only native and conformationally intact lactoferrin from bovine or human milk, colostrum, or serum could completely block HCMV infection”. In vivo, effects of LF include anti-tumor (Bezault et al., Cancer Research 54:2310-2312, 1994), anti-viral (Shimizu et al., Arch. Virol. 141:1875-1889, 1996) and antimicrobial activities (Trumpler et al., Eur. J. Clin. Microbiol. Infect. Dis. 8:310-313, 1989; Zagulski et al., Br. J. Exp. Pathol. 70:697-704, 1989).

Lactoferrin interferes with processes of host-bacterial interaction and can affect some important factors of virulence of S. aureus (Staphylococcus aureus) such as cell growth rate, morphology and ultrastructure (Diarra, et al., J. Dairy Sci 85:1141-1149, 2002). Lactoferrin has been considered to play a role in immunomodulation and transcriptional activation of various molecules (He and Furmanski, Nature 373:721-724, 1995; Kanyshkova, et al., FEBS letters 451:235-237, 1999). Furthermore, a synergism between LF and β-lactam antibiotics on bacterial growth inhibition of all four classes of β-lactamase-producing S. aureus strains has been reported (Diarra et al., supra).

Lactoferrin is usually purified from milk or milk whey (i.e. lactoserum) by one or more of the following types of column chromatography: ion-exchange, especially cation-exchange; affinity (viz. immobilized heparin, single-stranded DNA, lysine or arginine); dye-affinity; and size exclusion. Membrane ultrafiltration can also be used to separate lactoferrin from milk or whey. An industrial process for lactoferrin purification which employs both cation-exchange chromatography and tangential-flow membrane filtration is described by Tomita et al. (Biochem. Cell Biol. 80:109-112, 2002). Details of lactoferrin purification using cation-exchange chromatography are given by Okonogi et al. (New Zealand Patent No. 221,082), Ulber et al. (Acta Biotechnol. 21:27-34, 2001) and Zhang et al. (Milchwissenschaft 57: 614-617, 2002). None of these processes, nor any other existing process for commercial-scale purification of lactoferrin, are able to effectively remove contaminants that affect the stability and/or activity of lactoferrin. Examples of such contaminants may be contaminating protease(s) or proteolytic degradation fragments of lactoferrin.

Several workers have used a surfactant in conjunction with hydrophobic interaction chromatography (HIC) to purify proteins other than lactoferrin. Of particular relevance, both Wetlaufer and Koenigbauer (Wetlaufer, D. B. & Koenigbauer, M. R., J. Chromatogr. 359:55-60, 1986), and Rukhadze et al. (Rukhadze, M. D., et al., Biomed. Chromatogr. 17:538-542, 2003), showed that surfactants can change the retention behavior of proteins on hydrophobic adsorbents.

Three independent references presented below describe the behaviour and/or purification of lactoferrin when subjected to HIC. Two patents describe this type of chromatography to separate human lactoferrin from bovine lactoferrin by differential elution (U.S. Pat. No. 5,861,491 & New Zealand Patent No. 336 981). Cho et al. (Cho, J.-K., et al., Biosci. Biotechnol. Biochem. 64:633-635, 2000) describe lactoferrin purification by use of a salt gradient with a hydrophobic interaction resin. These workers specified that it was necessary for non-ionic surfactants to be present to ensure lactoferrin solubility and to separate it from human milk fat globule membrane, but did not contemplate that the hydrophobic nature of the resin employed was modified by the surfactants.

Finally, Machold et al. (J. Chromatogr. A972:3-19, 2002) describe the retention behaviour of bovine lactoferrin on several hydrophobic interaction resins under a range of salt concentrations.

Hydrophilic interaction chromatography (HILIC), a term introduced by Alpert in 1990 (J. Chromatogr. 499:177-196, 1990), is an entropically driven separation method that is mechanistically closely related to hydrophobic interaction chromatography (Gagnon, Expand your processing options with hydrophilic interaction chromatography, www.validated.com/revalbio/pdffiles/hilic.pdf, 1998; accessed Apr. 27, 2006). According to this source, in the presence of a moderate or greater concentration of a so-called “excluded solute”, and “a very strongly hydrated solid phase in the form of a chromatography support, proteins will preferentially share their hydration shells with the solid phase rather than with one another. They adsorb to the chromatography support. This process, driven by high concentrations of excluded solutes, is HILIC. The proteins can then be selectively eluted at high resolution in a descending gradient of the excluded solute.” Excluded solutes include salts such as ammonium sulphate, sodium sulphate and potassium phosphate and polyethylene glycol (PEG). All excluded solutes are kosmotropes, either charged or neutral, which tend to increase the structure of water and thereby cause the surfaces of proteins and solids to be preferentially hydrated (ibid.; cf. Collins and Washabaugh, Quart. Rev. Biophys. 18:323-422, 1985).

HILIC has experienced extensive development for purification of peptides in aqueous-organic solvent systems (reviewed in Yoshida, J. Biochem. Biophys. Methods 60:265-280, 2004). However, it has not been much used for protein purification since the pioneering work of Rubinstein (cf. Rubinstein, Anal. Biochem. 98:1-7, 1979). Aside from Rubinstein's work with high concentrations of n-propanol, there appears to be only one other published description of the use of HILIC to purify a protein, specifically a mouse IgM antibody adsorbed to an “oligo-polyethylene glycol” chromatography resin in the presence of 10% PEG of molecular weight 6,000 daltons (Gagnon, Purification Tools for Monoclonal Antibodies, Validated Biosystems, Inc., Tucson, Ariz., USA, 1996, Ch. 8). Although the same author (Gagnon, op. cit., 1998) advocates the practice of this protein purification technology using chromatographic supports including Superdex™ (GE Healthcare) and Toyopearl® Ether (Tosoh Bioscience), no description is given of the reduction of same to practice to purify any specific protein.

It appears that contaminant enzymes are present in currently existing commercial lactoferrin preparation. These enzymes are co-purified during lactoferrin purification from milk or lactoserum.

There is therefore a great need for new purification and stabilization methods of lactoferrin preparations in order to remove contaminating protease(s) or proteolytic degradation fragments in order to enhance, maintain or preserve the protein stability and activity of lactoferrin, for a longer period of time.

### SUMMARY OF THE INVENTION

It is one aim of the present invention to provide a process to remove enzyme contaminant responsible for lactoferrin degradation. Therefore, removal of these enzymes or addition of specific inhibitors would prevent degradation of a lactoferrin preparation and loss of activity of lactoferrin.

It is another aim of the present invention to provide a process to remove inhibitor of lactoferrin activity.

It is a further aim of the present invention to provide a process to enhance stability and/or improve activity of lactoferrin with respect to currently commercially available sources of lactoferrin.

In accordance with the present invention, there is provided a method for lactoferrin purification comprising the steps of contacting in a flowthrough mode a solution of lactoferrin, with a hydrophilic adsorbent in the presence of an excluded solute; and collecting a fraction containing lactoferrin substantially free of contaminant enzyme and/or lactoferrin inhibitor.

In accordance with the present invention, there is provided a method for purifying lactoferrin comprising the steps of contacting in a bind-and-elute mode and in an adsorptive fashion a solution of lactoferrin, with a hydrophilic adsorbent in the presence of an excluded solute, applying a decreasing concentration gradient of the excluded solute, and collecting a fraction containing lactoferrin substantially free of contaminant enzyme and/or lactoferrin inhibitor.

In accordance with the present invention, there is provided a method for purifying lactoferrin comprising the steps of contacting in a flowthrough mode a solution of lactoferrin, with a hydrophobic adsorbent in the presence of a surfactant, and in the presence of an excluded solute, and collecting a fraction containing lactoferrin substantially free of contaminant enzyme and/or lactoferrin inhibitor.

In accordance with the present invention, there is provided a method for purifying lactoferrin comprising the steps of contacting in a bind-and-elute mode and in an adsorptive fashion a solution of lactoferrin, with a hydrophobic adsorbent in the presence of a surfactant, and in the presence of an excluded solute, applying a decreasing concentration gradient of the excluded solute, and collecting a fraction containing lactoferrin substantially free of contaminant enzyme and/or lactoferrin inhibitor.

In another embodiment of the present invention, it is provided the method of the present invention, wherein the excluded solute is selected from the group, consisting of, but not limited to, ammonium sulfate, sodium sulfate, potassium phosphate and sodium chloride.

In addition, the surface of the hydrophilic adsorbent is substantially composed of one or more of the following substances: agarose, dextran, poly(methyl methacrylate), polyacrylamide, poly(methoxyethylacrylamide), poly(vinyl alcohol), cellulose, carboxymethylcellulose, phenol/formaldehyde co-polymer, chitin or chitosan.

In another embodiment, the surface of the hydrophilic adsorbent is comprises polar functional groups.

In a further embodiment, the polar functional groups are either uncharged or bear charge.

In another embodiment, the surface of the hydrophilic adsorbent is substantially composed of silica, calcium silicate, hydroxyapatite, titanium dioxide or zirconia.

In addition, the surface of the hydrophilic adsorbent is substantially composed of a mineral.

In accordance with the present invention, the surface of the hydrophilic adsorbent is substantially composed of a hydrophobic substance which is substantially covered, either by coating or chemical bonding, with one or more polar substances so as to render it hydrophilic.

Further, the surface of the hydrophobic adsorbent is substantially composed of polystyrene/divinyl benzene co-polymer.

In another embodiment of the present invention, it is provided a method as defined in the present invention, wherein the surface of the hydrophobic adsorbent is substantially composed of a hydrophilic substance which is substantially covered, either by coating or chemical bonding, with one or more non-polar substances so as to render it hydrophobic.

In a further embodiment, the surfactant is a nonionic, anionic, cationic or zwitterionic surfactant.

In addition, most preferably the surfactant is Polysorbate 20, Tween™ 20, Tween™ 80 or Tergitol™ NP-9.

In another embodiment, the hydrophilic adsorbent is Superdex, Sephadex, Superose, Sephacryl, Sepharose, cross-linked agarose, a Toyopearl® size exclusion protein chromatography medium, Toyopearl® Ether or Fractogel® EMD BioSEC.

In a further embodiment, the hydrophobic adsorbent is Phenyl Sepharose.

In another embodiment, it is provided a method according to the present invention, further comprising filtering the solution of lactoferrin before adsorbing same to the hydrophobic and/or hydrophilic adsorbent.

In addition, it is provided a method for purifying lactoferrin as defined in the present invention wherein the lactoferrin is purified from, milk, lactoserum or from a source of recombinant lactoferrin.

Preferably, the solution of lactoferrin is milk.

In accordance with the present invention, there is provided a method for purifying lactoferrin comprising the steps of adsorbing a solution of lactoferrin to a hydrophobic adsorbent in the presence of an aqueous acidic solution containing concentration of a charged excluded solute, applying an increasing pH gradient and collecting a fraction containing lactoferrin substantially free of contaminant enzyme and/or lactoferrin inhibitor by applying a decreasing concentration gradient of the excluded solute.

In a further embodiment, the adsorbent is a phenyl-functional hydrophobic interaction chromatography medium selected from the group consisting of Phenyl Sepharose HP, Phenyl Sepharose Fast Flow (low substitution), Phenyl Sepharose Fast Flow (high substitution), Toyopearl® Phenyl-650, TSKgel Phenyl-5PW, Fractogel® EMD Phenyl 650 and Poros HP2.

In a further embodiment, the adsorbent is a hydrophobic interaction resin, other than a polyethylene glycol-functional resin, the surface of said hydrophobic interaction resin being substantially composed of alkyl and/or aryl functional groups.

In a further embodiment, the aqueous solution is a buffer solution.

According to the present invention, the pH of the aqueous acidic solution is between 3.0 and 4.5. Most preferably, the pH of the aqueous acidic solution is 3.8.

In an addition embodiment, it is provided a method for stabilizing lactoferrin in an enzyme-containing lactoferrin solution comprising the step of adding at least one enzyme inhibitor to said solution for reducing enzymatic degradation of lactoferrin.

In a further embodiment, the enzyme inhibitor is a protease inhibitor. Most preferably the enzyme inhibitor is aspartyl or serine protease inhibitor.

In accordance with the present invention, there is provided a lactoferrin substantially free of contaminant enzyme and/or lactoferrin inhibitor.

In accordance with the present invention, there is provided a lactoferrin substantially free of contaminant enzyme and/or lactoferrin inhibitor, said lactoferrin being produced by the method as defined in the present invention.

In a further embodiment, it is provided a lactoferrin comprising at least one enzyme inhibitor preventing degradation of lactoferrin.

In a further embodiment, it is provided a lactoferrin substantially free of contaminant enzyme, said lactoferrin being stable in solution retaining its activity.

In a further embodiment, it is provided a lactoferrin substantially free of contaminant enzyme, said lactoferrin remaining stable in solution for at least 6 months.

In addition, it is provided a lactoferrin as defined in the present invention, having a purity of at least 95%.

In addition, it is provided a lactoferrin as defined in the present invention, having a minimal inhibitory concentration of at least 1 mg/ml.

In a further embodiment, it is provided a stabilized lactoferrin comprising at least one enzyme inhibitor preventing degradation of lactoferrin.

In a further embodiment, it is provided a stabilized lactoferrin as defined in the present invention, having more than 89% growth inhibitory activity on S. aureus.

### BRIEF DESCRIPTION OF THE DRAWINGS

FIG. 1 illustrates a SDS-PAGE gel electrophoresis of bovine lactoferrin (25 μg; Commassie blue staining) obtained from different suppliers and subjected or not to dialysis;

FIG. 2 illustrates a SDS-PAGE gel electrophoresis of bovine lactoferrin (25 μg; Commassie blue staining) obtained from different suppliers and different milk sources prior to purification;

FIG. 3 represent a SDS-PAGE gel electrophoresis (5-14%, 6.5 μg/well; Silver staining) of a commercial lactoferrin (LFnp) incubated over 4 days with (+) or without (−) serine protease inhibitor (AEBSF; 1 mM);

FIG. 4 represent a SDS-PAGE gel electrophoresis (5-14%, 6.5 μg/well; Silver staining) of a commercial lactoferrin (LFnp) incubated over 7 days with (+) or without (−)1 μM of Pepstatin A (10 μM). FIG. 4a represents the incubation at 4° C., FIG. 4b illustrates the incubation at room temperature and FIG. 4c illustrates the incubation at 37° C.;

FIG. 5 is a chromatogram showing absorbance (1) at 280 nm and conductivity (2) in accordance with Example 3 of bovine lactoferrin (10 mg/ml) purified by flowthrough purification on Superdex™ 200 (Run 05CR1-HIC31);

FIG. 6 is a chromatogram showing absorbance at 280 nm (1) and conductivity (2) in accordance with Example 4 of bovine lactoferrin (10 mg/ml) purified by bind-and-elute purification on Superdex™ 200 (Run 05CR1-HIC30);

FIG. 7 is a chromatogram showing absorbance at 280 nm (1) and conductivity (2) in accordance with Example 5 of bovine lactoferrin (10 mg/ml) purified by flowthrough purification on Sephacryl™ S-400 (Run 05CR1-HIC50) according to Example 5;

FIG. 8 is a chromatogram showing absorbance at 280 nm (1) and conductivity (2) in accordance with Example 6 of bovine lactoferrin (10 mg/ml) purified by bind-and-elute purification on Sephacryl™ S-400 (Run 05CR1-HIC51);

FIG. 9 is a chromatogram showing absorbance at 280 nm (1) and conductivity (2) in accordance with Example 7 of bovine Euro Protein lactoferrin (10 mg/ml) purified by bind-and-elute purification on Sephacryl™ S-400 (Run 05CR1-HIC56);

FIG. 10 is a chromatogram showing absorbance at 280 nm (1) and conductivity (2) in accordance with Example 8 of bovine lactoferrin (10 mg/ml) purified by bind-and-elute purification on SP Sepharose™ (Run 05CR1-HIC55);

FIG. 11 is a chromatogram showing absorbance at 280 nm (1) and conductivity (2) in accordance with Example 9 of bovine lactoferrin (10 mg/ml) purified by flowthrough purification on Toyopearl® Ether (Run 05CR1-HIC28);

FIG. 12 is a chromatogram showing absorbance at 280 nm (1) and conductivity (2) in accordance with Example 10 of bovine lactoferrin (10 mg/ml) purified by bind-and-elute purification #1 on Toyopearl® Ether (Run 05CR1-HIC37);

FIG. 13 is a chromatogram showing absorbance at 280 nm (1) and conductivity (2) in accordance with Example 11 of bovine lactoferrin (10 mg/ml) purified by bind-and-elute purification #2 on Toyopearl® Ether (Run 05CR1-HIC29);

FIG. 14 is a chromatogram showing absorbance at 280 nm (1) and conductivity (2) in accordance with Example 12 of bovine lactoferrin (10 mg/ml) purified by bind-and-elute purification on Phenyl Sepharose™ HP (Run 05CR1-HIC40);

FIG. 15 is a chromatogram showing absorbance at 280 nm (1), percentage of buffer B (2) and conductivity (3) in accordance with Example 13 of bovine lactoferrin (10 mg/ml) purified by bind-and-elute purification on Phenyl Sepharose™ HP (Run 05CR1-HIC27);

FIG. 16 is a chromatogram showing absorbance at 280 nm (1), percentage of buffer B (2) and conductivity (3) in accordance with Example 14 of bovine lactoferrin (20 mg/ml) purified by bind-and-elute purification on Phenyl Sepharose™ HP (Run 3.4-L Phenyl Sepharose R1);

FIG. 17 is a SDS-PAGE gel electrophoresis of bovine lactoferrin (20 mg/ml; Silver staining; 6.5 μg) purified on Phenyl Sepharose™ HP resin in accordance with Example 14;

FIG. 18 is a SDS-PAGE gel electrophoresis of bovine lactoferrin (20 mg/ml; Silver staining; 6.5 μg) purified on Phenyl Sepharose™ HP resin in accordance with Example 14;

FIG. 19 is an HPLC result of bovine lactoferrin (20 mg/ml) purified on Phenyl Sepharose™ HP resin in accordance with Example 14 (run on 3.4-L Phenyl Sepharose);

FIG. 20 is a chromatogram showing absorbance at 280 nm (1), pH (2) and conductivity (3) in accordance with Example 15 of bovine lactoferrin (10 mg/ml) purified by bind-and-elute purification on Phenyl Sepharose™ HP (Run 05CR1-HIC45);

FIG. 21 is a SDS-PAGE gel electrophoresis of bovine lactoferrin (10 mg/ml; Silver staining; 6.5 μg) purified by bind-and-elute purification on Phenyl Sepharose HP in accordance with Example 16;

FIG. 22 is a SDS-PAGE gel electrophoresis of bovine lactoferrin (20 mg/ml; Silver staining; 6.5 μg) purified on Phenyl Sepharose™ HP resin in accordance with Example 14 and subjected to different incubation times in pH 7.2 solution buffer at 4° C.;

FIG. 23 is an SDS-PAGE gel electrophoresis of bovine lactoferrin (20 mg/ml; Silver staining; 6.5 μg) purified on Phenyl Sepharose™ HP resin in accordance with Example 14 and subjected to different incubation times in pH 7.2 solution buffer at 30° C.;

FIG. 24 is an SDS-PAGE gel electrophoresis of bovine lactoferrin (106 mg/ml; Commassie blue staining; 2 μg) purified on Phenyl Sepharose™ HP resin in accordance with Example 14 and maintaining product stability in solution up to 6 months at room temperature;

FIG. 25 is an SDS-PAGE gel electrophoresis of non-purified bovine lactoferrin (107 mg/ml; Commassie blue staining; 2 μg) and showing evidence of product instability in solution at room temperature;

FIG. 26 is an SDS-PAGE gel electrophoresis of non-purified bovine lactoferrin (107 mg/ml; Silver staining; 2 μg) and showing extensive evidence of product instability in solution at room temperature;

FIG. 27A is the minimal inhibitory concentrations (MIC) determined by broth microdilution of bovine lactoferrin purified on Phenyl Sepharose™ HP resin (LFp) in accordance with Example 14 compared to commercial lactoferrin preparations (LFnp) obtained from DMV International (FIG. 27B), Morinaga (FIG. 27C), Euro Protein (FIG. 27D) and Glanbia (FIG. 27E);

FIG. 28 is the minimal inhibitory concentrations (MIC) determined by broth microdilution of commercial non-purified lactoferrin (LFnp) from DMV International versus commercial lactoferrin preparations (LFnp) obtained from Biopole and either extracted from milk or lactoserum;

FIG. 29 is an SDS-PAGE gel electrophoresis (Silver staining, 2 and 6.5 μg) of bovine lactoferrin (106 mg/ml) purified on Phenyl Sepharose™ HP resin in accordance with Example 14, non-purified lactoferrin from DMV (107 mg/ml), non-purified lactoferrin extracted from milk by Biopole (110 mg/ml) or non-purified lactoferrin extracted from lactoserum by Biopole (124 mg/ml);

FIG. 30 illustrates the effect of purified bovine lactoferrin (LF Pure) on Phenyl Sepharose™ HP resin in accordance with Example 14 as compared to non purified bovine lactoferrin (LF non Pure) on somatic cell count (SCC) response in milk of 6 cows infused in different quarters of the mammary gland with buffer, 1 g of LF Pure, 1 g of LF non Pure or no infusion; and

FIG. 31 is a silver stained 2D-PAGE separation (pH 6-11) of lactoferrin purified on Phenyl Sepharose™ HP resin in accordance with Example 14. Numbers indicate spots cut-out from the gel and subjected to digestion with a sequence grade trypsin for subsequent proteomic analysis.

### DETAILED DESCRIPTION OF THE PREFERRED EMBODIMENTS

While the making and using of various embodiments are discussed below, it should be appreciated that the specific embodiments discussed herein are merely illustrative of specific ways of making and using the invention and should not be construed as to limit the scope of the invention.

It should also be appreciated that the processes described herein in accordance with specific embodiments of the present invention are not limited to a specific source of lactoferrin, but can be applied to any source of lactoferrin, including, but not limited to, human lactoferrin.

It has now been found that existing lactoferrin contains contaminants responsible for protein degradation and decrease of lactoferrin activity. It is not known in the prior art any source of lactoferrin currently having sustained protein stability in solution. Consequently, the lactoferrin that one will obtain by purifying it in accordance with the present invention will have and/or retain its protein stability in solution longer than any other source of lactoferrin currently available.

Using lactoferrin as therapeutics in particular requires purity in excess of 95% and long term stability especially in solution, for storage consideration.

In accordance with the present invention, there is provided a method for lactoferrin purification employing hydrophilic interaction chromatography (HILIC) in the presence of excluded solutes, as described hereinafter, for maintaining protein stability in solution and preserving lactoferrin activity.

In another embodiment of the present invention, in an alternative for purifying lactoferrin described in detail hereinafter, lactoferrin is purified by chromatography on Superdex™ 200 resin (GE Healthcare) in the presence of moderate or greater concentrations of ammonium sulphate. This chromatography matrix is described by the manufacturer as comprising a spherical composite of cross-linked agarose and dextran. Although some purification is achieved in flowthrough mode (Example 3), a preferred embodiment with this resin employs bind-and-elute, or adsorption, mode (Example 4).

Although the present invention contemplates neutral excluded solutes in general for lactoferrin purification by hydrophilic interaction chromatography, polyethylene glycol of molecular weight 6,000 daltons (PEG-6000) is not a preferred excluded solute because lactoferrin is only poorly soluble in the presence of moderate concentrations of this solute. For example, bovine lactoferrin dissolves only to an extent of approximately 0.3 mg/ml in 20 mM sodium phosphate, pH 7, containing 10% w/v PEG-6000.

In a further embodiment of the present invention, in an alternative method for purifying lactoferrin described in detail hereinafter, lactoferrin is purified by chromatography on Sephacryl™ S-400 resin (GE Healthcare) in the presence of moderate or greater concentrations of ammonium sulphate. This chromatography matrix is described by the manufacturer as comprising spherical alkyl dextran and N,N′-methylenebisacrylamide. Although some purification is achieved in flowthrough mode (Example 5), a preferred embodiment with this resin employs bind-and-elute, or adsorption, mode (Examples 6 and 7).

In another embodiment, an alternative method for purifying lactoferrin is disclosed hereinafter. Lactoferrin is purified by chromatography on SP Sepharose™ resin (GE Healthcare) in the presence of a high concentration of ammonium sulphate. This chromatography matrix is described by the manufacturer as comprising a highly cross-linked agarose matrix to which is coupled strongly cationic sulfopropyl groups. It is shown that by use of a high concentration of an ionic excluded solute such as ammonium sulfate, chromatography resins bearing a fixed negative charge can be used to purify lactoferrin through the practice of the present invention. A person skilled in the art would acknowledge, in light of the teaching of the present invention which discloses methods employing essentially uncharged resins, that the nature of the fixed charge(s) on the resin is substantially irrelevant to the practice of the invention. Therefore the novel method for purifying lactoferrin described herein contemplates use of either chromatography resins bearing no fixed charge, or those bearing fixed charges of either or both signs. Further, one skilled in the art will be able to determine the concentration of the ionic excluded solute necessary for lactoferrin purification without undue experimentation or teaching.

In another embodiment, it is disclosed a method for purifying lactoferrin, by chromatography on Toyopearl® Ether resin (Tosoh Bioscience) in the presence of moderate or greater concentrations of ammonium sulphate. This chromatography matrix is described by the manufacturer as comprising a hydrophilic polymer resin matrix to which are coupled polyethylene glycol chains.

It is necessary to clearly distinguish the present invention disclosing alternative methods of lactoferrin purification by hydrophilic interaction chromatography (HILIC) from those methods of the prior art using hydrophobic interaction chromatography (HIC). Two lines of argument will be followed in order to demonstrate this difference, specifically: the properties relative to protein adsorption of polyethylene glycol (PEG) surfaces; and the teaching of the prior art in regard to the chromatographic application of such surfaces for protein purification by HIC.

It is justifiable to contend that PEG surfaces are predominantly hydrophilic in character, possessing only secondary properties of a hydrophobic nature, and that the balance of these opposing tendencies in favour of the former generally discourages protein adsorption. As stated by Poole et al. (J. Chromatogr. A 898: 211-226, 2000), “In general, one can state that the poly(ethylene glycol) stationary phases are strongly dipolar/polarizable and hydrogen-bond basic . . . . Dispersion interactions also make an important contribution to retention” (cf. West and Lesellier, Chromatogr. A 1110: 200-213, 2006). Although it has been asserted that “poly(ethylene glycols) are hydrophobic in nature and will interact favourably with the hydrophobic side chains [of proteins] exposed upon unfolding” (Lee and Lee, Biochemistry 26:7813-7819, 1987), the overwhelming consensus of scientific opinion is that proteins interact either unfavourably or not at all with PEG. Thus the same authors state that “polyethylene glycol is excluded from the protein domain . . . the present study has demonstrated that the induced phase separation of proteins from PEG-water system is due to the unfavourable thermodynamic interaction between protein and PEG” (Lee and Lee, J. Biol. Chem. 256:625-631, 1981; cf. Atha and Ingham, J. Biol. Chem. 256:12108-12117, 1981).

Furthermore, as stated by Karlström and Engkvist, “Quite frequently PEG is used to coat surfaces in order to prevent proteins or other macromolecular material from depositing on the surface. The general mechanism behind the effective repulsion of the macromolecule from the coated surface is that if the macromolecule comes close to the surface, then the conformational degrees of freedom of the polymer are drastically reduced, and this causes an entropic repulsion between the surface and the macromolecule” (Karlström and Engkvist, Theory of poly[ethylene glycol] in solution, Ch. 2, in Harris, J. M., Ed., Poly[ethylene glycol]: Chemistry and biological applications, American Chemical Society, Washington, D.C., 1997). Finally, as stated by Gagnon, a knowledgeable and renowned person skilled in the art of hydrophilic interaction protein chromatography, “Doing ‘HIC’ on these [PEG] supports has actually been HILIC all along. They don't have enough hydrophobicity to affect selectivity that much. This is why their selectivity is so different from strongly hydrophobic supports like butyl, octyl, and phenyl” (Gagnon, op. cit., 1998).

Without being bound by theory, the prior art teaches away from the application of polyethylene glycol-functional resins for purification of lactoferrin by hydrophobic interaction chromatography. Specifically, none of the references supra describing purification of lactoferrin by HIC employed a PEG-functional resin, such as Toyopearl® Ether. Moreover, “TOYOPEARL® Ether is recommended for the purification of very hydrophobic proteins such as monoclonal antibodies or membranes proteins” (Hydrophobic Interaction Chromatography, Product Brochure, Tosoh Bioscience, Montgomeryville, Pa., USA). That lactoferrin is not particularly hydrophobic is demonstrated by its substantial solubility in 2.3 M ammonium sulphate (Example 8), as well as by the fact that it is soluble in neutral buffers to a concentration of at least 100 mg/ml. Finally, given that bovine lactoferrin was ranked in terms of hydrophobicity as fourth out of six common proteins (Machold et al., op. cit., 2002), it is evidently not a candidate for purification by HIC on a PEG-functional resin. This is reflected in the fact that these authors did not include a PEG-functional resin amongst the 15 chromatographic sorbents that they selected for their HIC protein selectivity comparison.

In one embodiment of the present invention, it is disclosed an alternative method for purifying lactoferrin by chromatography on Phenyl Sepharose™ HP resin (GE Healthcare) whose surface has been previously modified by adsorption of a non-ionic surfactant (i.e. Tween™ 20, Polysorbate 20), again in the presence of moderate or greater concentrations of ammonium sulphate. Such modification of the surface of HIC resins by surfactants is well known in the art, and the relevant phenomenon has been described as follows: “In fact, the surface layer on the stationary phase is formed as a result of hydrophobic interaction between long alkyl chain of Tween-80™ and phenyl groups of stationary phase. Thus, the alkyl chain of Tween-80™ interacts with phenyl groups, but hydrophilic polyoxyethylene groups remain directed from the stationary phase towards the mobile phase” (Rukhadze et al., Biomed. Chromatogr. 17:538-542, 2003). Although such conversion of a hydrophobic absorbent into an effectively hydrophilic adsorbent is not novel, explicit use of the latter in a hydrophilic interaction chromatographic process for lactoferrin purification is novel and inventive. Thus it will be evident to those skilled in the art that Examples 12 to 14 represent another embodiment of the present invention, namely lactoferrin purification by HILIC, differing only in details from Examples 9 to 11.

In another embodiment of the present invention, in the practice of the invention employing a hydrophobic sorbent in the presence of a surfactant, it is preferable that the latter be present at a concentration at or above its critical micelle concentration (CMC) in order to provide adequate coverage of the sorbent surface with surfactant. With reference to Examples 12 to 14 employing Tween™ 20, it has been determined that the CMC of this surfactant under the conditions of these experiments is on the order of 1 mg/L.

In a further embodiment, the surfactant used to modify the hydrophobic surface of the chromatography resin need not be non-ionic in nature. In particular, the present invention discloses the possibility that any of non-ionic, anionic, cationic or zwitterionic surfactants can be used, alone or in combination, as additives to convert a substantially hydrophobic surface into a substantially hydrophilic one for the novel practice of lactoferrin purification by hydrophilic interaction chromatography.

In another embodiment, it is disclosed a method for purifying lactoferrin, wherein lactoferrin adsorbs to the surface of a HIC resin under acidic pH conditions in the presence of a salt concentration. Purified lactoferrin is eluted at a higher pH by contacting the lactoferrin-adsorbed resin with a solution containing a lower salt concentration.

Finally, the present invention provides for the first time a novel lactoferrin with protein stability in solution never reported nor seen in commercial preparations currently available on the market. Further, the stability of said lactoferrin can be retained for at least 6 months at room temperature in solution.

It is believed that the presence of contaminant bands in lactoferrin preparation might be due to co-purification of other milk peptides and proteins such as enzymes and (or) the results of enzymatic degradation when lactoferrin is put into solution. To verify the presence of enzymes in commercial lactoferrin preparation, the effect of different enzyme inhibitors was tested.

The present invention further discloses that the degradation of commercial lactoferrin preparation is inhibited by selected enzyme inhibitors such as protease inhibitors (e.g. aspartyl protease or serine protease inhibitors).

In one embodiment of the invention, the surfactant used in the method of the present invention is a non-ionic surfactant, such as Polysorbate 20 or Tween™ 20, Tween™ 80 or Tergitol™ NP-9.

In one embodiment of the invention, the adsorbent used in the method of the present invention is Phenyl Sepharose. In a further embodiment, the method of the present invention further comprises filtering lactoferrin before adsorbing it to the hydrophobic adsorbent.

In a further embodiment of the invention, the first pH is between about 3.0 and 4.5, and more preferably about 3.8.

In one embodiment of the invention, the enzyme inhibitor is a protease inhibitor, specifically an aspartyl or serine protease inhibitor.

The lactoferrin as obtained from one embodiment of the method of the present invention has a purity of at least 95%. In a further embodiment, the lactoferrin obtained has a minimal inhibitory concentration of at least 1 mg/ml. Still in a further embodiment, the stabilized or purified lactoferrin solution obtained with the process of the present invention has more than 89% growth inhibitory activity on S. aureus.

As referred herein, the term “hydrophilic” in reference to the surface of a solid used for chromatography according to the practice of the invention means that the preponderant components forming the matrix of the solid are polar in nature, and/or that the matrix is effectively covered with polar functional groups. In this context, “polar” means that the isolated molecules from which the matrix or functional groups are normally formed have a dipole moment which is non-zero, in general of magnitude of at least one debye unit (cf. Dean, Lange's Handbook of Chemistry, 15^{th} edition, McGraw-Hill, New York, 1999, p. 5. 136).

As referred herein, the term “hydrophobic” in reference to the surface of a solid used for chromatography according to the practice of the invention means that the surface is not hydrophilic.

It is understood that the term “gradient” is meant to include a stepwise as well as a continuous (linear or non-linear) gradient.

The term “purity” is meant to be the amount of lactoferrin relative to the amount of total protein present, as determined by high performance liquid chromatography (HPLC) using U.V. detector or by any other method known in the art.

The term “non-purified lactoferrin” is meant to be lactoferrin extracted directly from either milk or lactoserum or other mediums in the case of recombinant lactoferrin and not subjected to additional purification steps.

In the present invention, the term “substantially” in the context of lactoferrin stability refers to a lactoferrin being free of degrading enzyme.

As referred herein, the term “fraction” means one or more fractions.

The expression “protein stability” refers to the presence of intact lactoferrin, meaning showing on a gel a decrease in the number of degradation fragments for a longer period of time than that which is currently seen in commercial preparations. The decreasing degradation of lactoferrin will induce a stability of the activity of the fractions since more intact lactoferrin protein is present.

The present invention would be readily understood by referring to the following Examples which are given to illustrate the invention rather than to limits its scope.

### Example 1

### Commercial Lactoferrin Purity

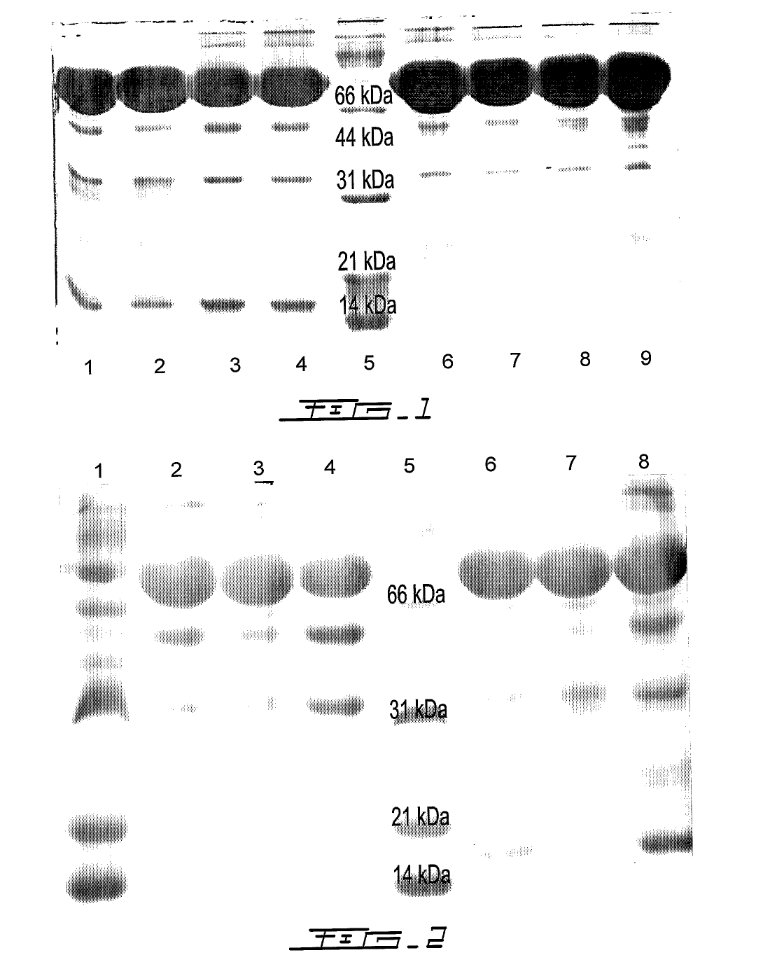

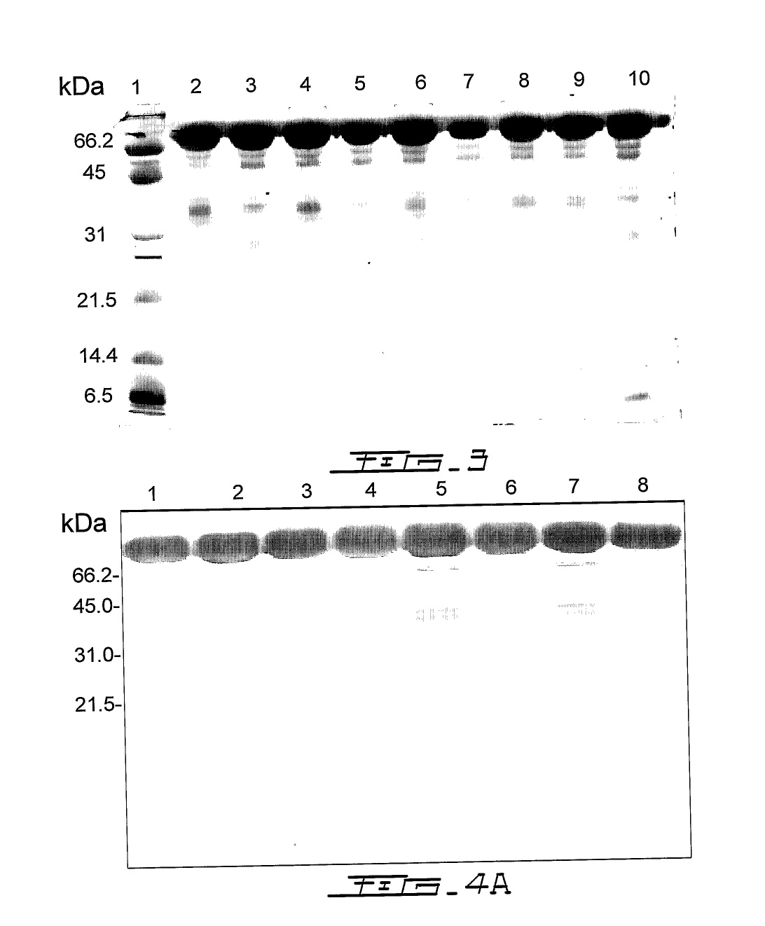

SDS-PAGE gel electrophoresis of bovine lactoferrin (25 μg) from different suppliers and sources are shown in FIGS. 1 and 2. In FIG. 1, the suppliers are DMV (lanes 1,2), Glanbia (lanes 3,4), Armor (lanes 6,7) and subjected (lanes 1, 3, 6) or not to dialysis (lanes 2, 4, 7), compared to a standard (lane 5) and LF150 (lane 8) and LF.45 (lane 9). In FIG. 2, the suppliers were Armor (lane 2), lactoferrin purified from milk (lane 3), lactoferrin purified from whey (lane 4), DMV (lanes 6, 7) and Glanbia (lane 8), and were compared to a standard (lanes 1, 5). In each of these products, there can be noticed several contaminant bands. Pure lactoferrin has a molecular weight expected of 78 kDa. Dialysis did not have a highly significant visual effect even though the intensity of the different SDS-PAGE bands was slightly enhanced in particular for the Armor product (FIG. 1, lanes 6,7; and FIG. 2, lane 2). In Example 18 hereinafter, it will be shown that this particular source of lactoferrin has a poor growth inhibitory activity against bacteria. LF150 (FIG. 1, lane 8) and LF.45 (FIG. 1, lane 9) are lactoferrin from Armor subjected to filtration with cut-off of 0.45 μm pore size and 150 kDa; only significant effect of this process on band profile is the removal of a band at about 21 kDa and the intensification of the other bands.

Low molecular weight contaminants (less than 21 KDa) are present in lactoferrin preparation extracted from raw milk and absent in preparation from whey (see lanes 2 vs. 3 in FIG. 2). On the other hand, fragments at about 50 and 35 kDa are more intense in preparation purified from lactoserum (FIG. 2, line 3) as compared to raw milk (FIG. 2, line 2), thus indicating a greater extent of product degradation before or during purification. This phenomenon will be further exemplified in Example 18

### Example 2

### Enzyme Inhibitors

The following enzyme inhibitors were tested according to manufacturer recommendations:

Plasmin inhibitor: 10 mM of lysine (Sigma);

Cysteine protease inhibitor: 10 μM of E64 (Sigma # E3132);

Aspartyl protease inhibitor: 10 μM of Pepstatin A (Sigma # P5318);

Serine protease inhibitor: 1 mM of AEBSF (Sigma # A8456).

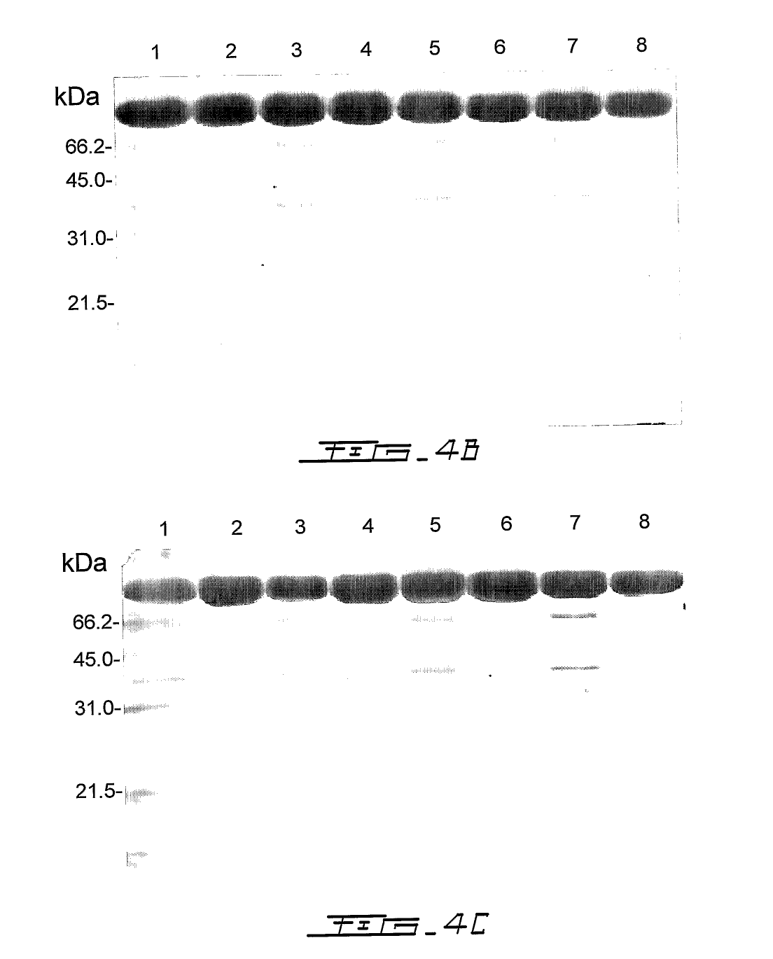

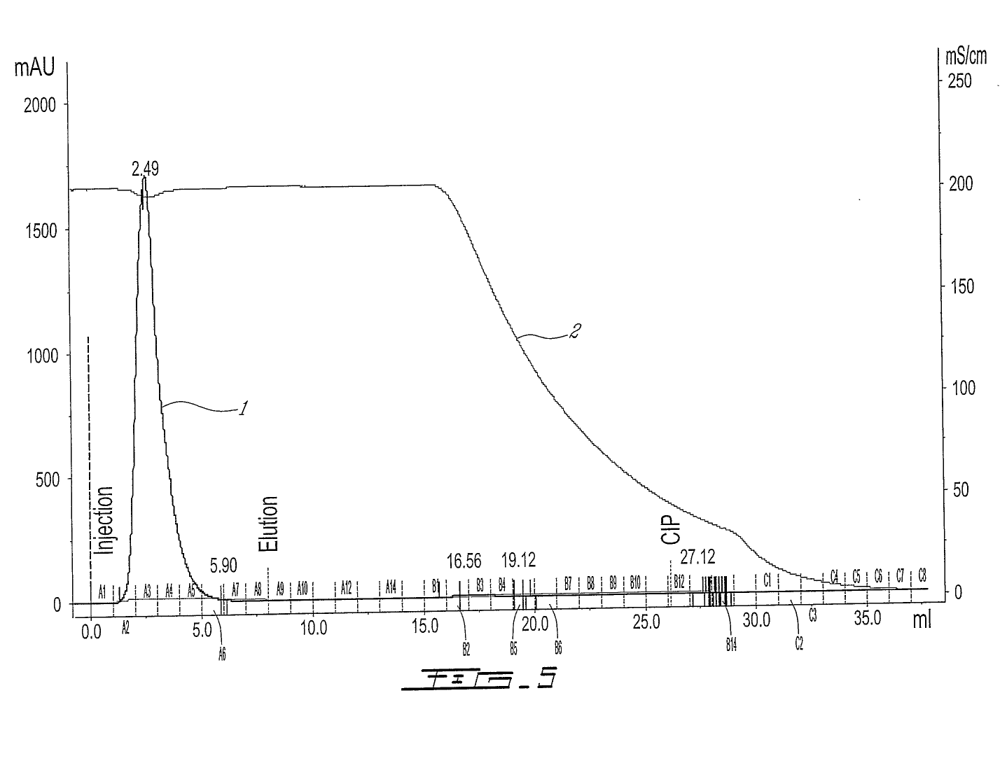

Commercial lactoferrin preparation (DMV International, Veghel, The Netherlands; Lot No. 10191343; approximately 92 percent pure by HPLC) was incubated (25 mg/ml; LFnp, FIGS. 3 and 4) for several days with or without inhibitor at 4° C. and 30° C. in the case of plasmin inhibitor or 4° C., room temperature or at 37° C. for all other inhibitors. After incubation, samples were frozen until SDS-PAGE analysis and subsequent Coomassie blue coloration or silver staining according to standard procedure. There was no effect of plasmin or cysteine protease inhibitors on SDS-PAGE band profile. However, appearance of degradation bands was inhibited by serine protease (FIG. 3) or aspartyl protease inhibitors (FIG. 4a, at 4° C.; FIG. 4b at room temperature; and FIG. 4c at 37° C.).

FIG. 3 illustrates the results of the commercial lactoferrin LFnp incubated over 4 days with or without a serine protease inhibitor (AEBSF). Lanes were loaded with:

| Product | Lane |
|---|---|
| Standard | 1 |
| LFnp without AEBSF incubated for 0 days. | 2 |
| LFnp without AEBSF incubated for 1 day. | 3 |
| LFnp with AEBSF incubated for 1 day. | 4 |
| LFnp without AEBSF incubated for 2 days. | 5 |
| LFnp with AEBSF incubated for 2 days. | 6 |
| LFnp without AEBSF incubated for 3 days. | 7 |
| LFnp with AEBSF incubated for 3 days. | 8 |
| LFnp with AEBSF incubated for 4 days. | 9 |
| LFnp without AEBSF incubated for 4 days. | 10 |

FIG. 4A, B, C illustrates the results of the commercial lactoferrin LFnp incubated over 7 days with or without Pepstatin A. Lanes were loaded with:

| Product | Lane |
|---|---|
| LFnp without Pepstatin A incubated for 1 day. | 1 |
| LFnp with Pepstatin A incubated for 1 day. | 2 |
| LFnp without Pepstatin A incubated for 2 days. | 3 |
| LFnp with Pepstatin A incubated for 2 days. | 4 |
| LFnp without Pepstatin A incubated for 5 days. | 5 |
| LFnp with Pepstatin A incubated for 5 days. | 6 |
| LFnp without Pepstatin A incubated for 7 days. | 7 |
| LFnp with Pepstatin A incubated for 7 days. | 8 |

These results show the presence of contaminant enzymes in commercial lactoferrin preparation. These enzymes are co-purified during lactoferrin purification.

### Example 3

### Flowthrough Purification on Superdex™ 200 (Run 05CR1-HIC31)

Partially purified bovine lactoferrin (DMV International, Veghel, The Netherlands; Product No. 4061455, Lot No. 10231167; approximately 92 percent pure by HPLC) was dissolved at a nominal concentration of 10 mg/ml in the following equilibration buffer: 20 mM sodium phosphate and 1.6 M ammonium sulfate, titrated with NaOH to pH 7.0. The apparently colloidal material present was removed by filtration through a membrane filter (Pall Acrodisc® Supor® 0.22-μm pore size).

The lactoferrin solution (1.0 ml) was applied at a flow rate of 0.5 ml/min to a 1.0 ml bed of Superdex™ 200 Prep Grade resin (GE Healthcare; 5 cm bed depth) previously equilibrated with the above buffer, collecting 1-ml fractions of column eluate.

The column was washed with equilibration buffer until the absorbance returned to baseline, then eluted with a solution of 20 mM sodium phosphate, pH 7.0, at the same flow rate.

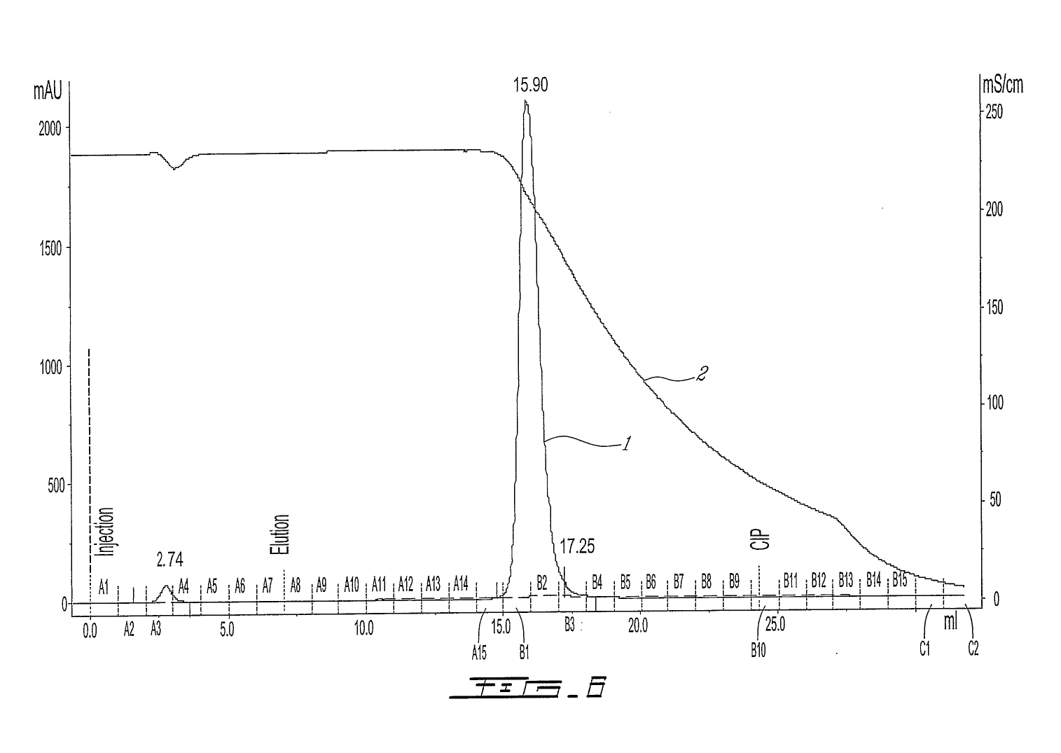

The resulting chromatogram (FIG. 5) shows that almost all of the applied protein passed through the resin in the flowthrough, apparently without adsorption.

Selected column eluate fractions were analyzed for lactoferrin content by HPLC (Table 1).

TABLE 1
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC31-S1 | feed | 12.38 | 1.0 | 12.38 | 94.26 | |
| 05CR1-HIC31-S2 | A1′ | 0.01 | 1.0 | 0.01 | 97.32 | 0.10 |
| 05CR1-HIC31-S3 | A2′ | 0.16 | 1.0 | 0.16 | 88.47 | 1.28 |
| 05CR1-HIC31-S4 | A3′ | 4.48 | 1.0 | 4.48 | 95.78 | 36.24 |
| 05CR1-HIC31-S5 | A4′ | 4.74 | 1.0 | 4.74 | 96.04 | 38.30 |
| 05CR1-HIC31-S6 | A5′ | 1.41 | 1.0 | 1.41 | 95.61 | 11.43 |
| 05CR1-HIC31-S7 | A6′ | 0.37 | 1.0 | 0.37 | 97.00 | 2.96 |
| 05CR1-HIC31-S17 | B1′ | 0.01 | 1.0 | 0.01 | 95.36 | 0.11 |
| 05CR1-HIC31-S18 | B2′ | 0.01 | 1.0 | 0.01 | 90.26 | 0.06 |
| 05CR1-HIC31-S19 | B3′ | 0.01 | 1.0 | 0.01 | 47.63 | 0.06 |
| | | | | Total | | 90.53 |
| | | | | recovery: | | |
| A3′-A6′ Pool: | % purity: | 95.9 | % | 88.9 | | |
| | | | recovery: | | | |

The fractions collected in the present invention are identified as A1′ to A15′, followed by B1′ to B15′.

The results show that the lactoferrin feed solution was somewhat increased in purity relative to the starting material, presumably due to the insolubility of one or more contaminants at the high concentration of ammonium sulfate employed. More importantly, the results show only a small increase in lactoferrin purity in a representative pool of fractions from the main peak.

Note that in Table 1, the fractions are designated with a prime (e.g. A1′) in recognition of the fact that due to an equipment malfunction each fraction is effectively offset to the left by 0.2-ml from the location shown on the chromatogram (FIG. 5). Thus, for example, the 1.0-ml fraction designated A1′ is that part of the chromatographic eluate commencing 0.2-ml following the start of fraction A1 on the Figure. This offset has no bearing on the purity or recovery results.

### Example 4

### Bind-and-Elute Purification on Superdex™ 200 (Run 05CR1-HIC30)

Partially purified lactoferrin was dissolved and purified exactly as in Example 3 except that the equilibration buffer consisted of 20 mM sodium phosphate and 2.0 M ammonium sulfate, titrated with NaOH to pH 7.0, and the lactoferrin feed solution was clarified using a Millex® GV, 0.22-μm pore size, membrane filter (Millipore).

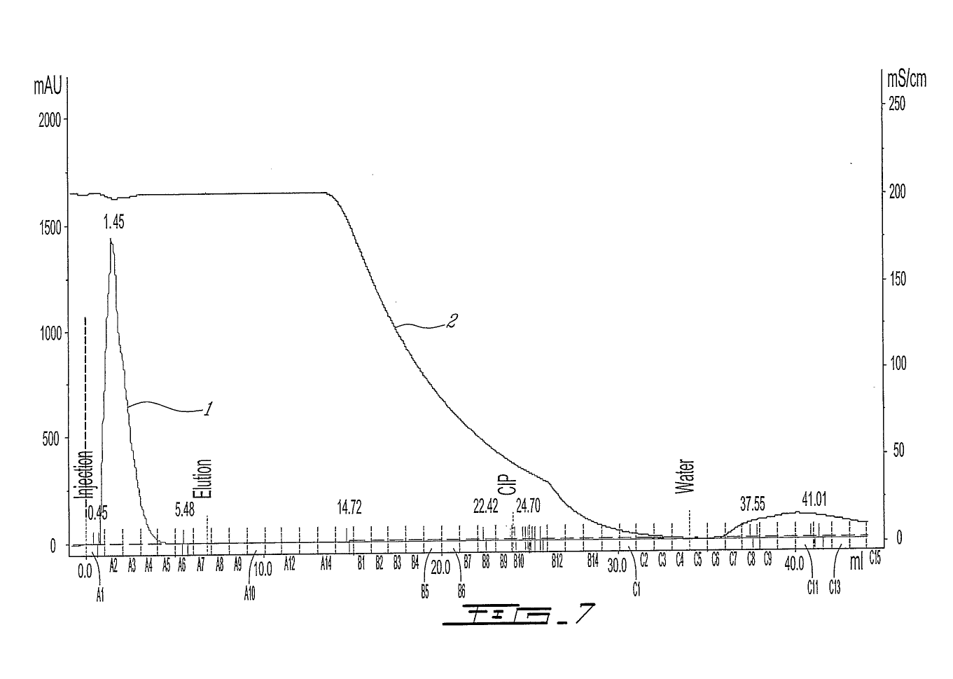

The resulting chromatogram (FIG. 6) shows that almost all of the applied protein adsorbed to the resin, and was eluted as the conductivity began to decrease from its initial value.

Selected fractions were analyzed for lactoferrin content by HPLC (Table 2).

TABLE 2
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC30-S1 | Feed | 8.24 | 1.0 | 8.24 | 95.27 | |
| 05CR1-HIC30-S2 | A2′ | 0.01 | 1.0 | 0.01 | 99.30 | 0.13 |
| 05CR1-HIC30-S3 | A3′ | 0.02 | 1.0 | 0.02 | 47.38 | 0.25 |
| 05CR1-HIC30-S4 | A15′ | 0.02 | 1.0 | 0.02 | 79.53 | 0.27 |
| 05CR1-HIC30-S5 | B1′ | 2.20 | 1.0 | 2.20 | 98.84 | 26.65 |
| 05CR1-HIC30-S6 | B2′ | 5.80 | 1.0 | 5.80 | 97.31 | 70.30 |
| | | | | Total recovery: | | 97.61 |
| B1′-B2′ Pool: | % purity: | 97.7 | % | 97.0 | | |
| | | | recovery: | | | |

The results show a substantial increase in lactoferrin purity to 97.7% in a representative pool of fractions from the main peak (i.e. B1′-B2′ pool), and with 97.0% recovery of the lactoferrin applied in the feed solution.

### Example 5

### Flowthrough Purification on Sephacryl™ S-400 (Run 05CR1-HIC50)

Partially purified lactoferrin was dissolved and purified as in Example 3 with the following exceptions: the chromatographic resin used was Sephacryl™ S-400 HR (GE Healthcare); and lactoferrin feed solution was clarified using a Millex® HV, 0.45-μm pore size, membrane filter (Millipore).

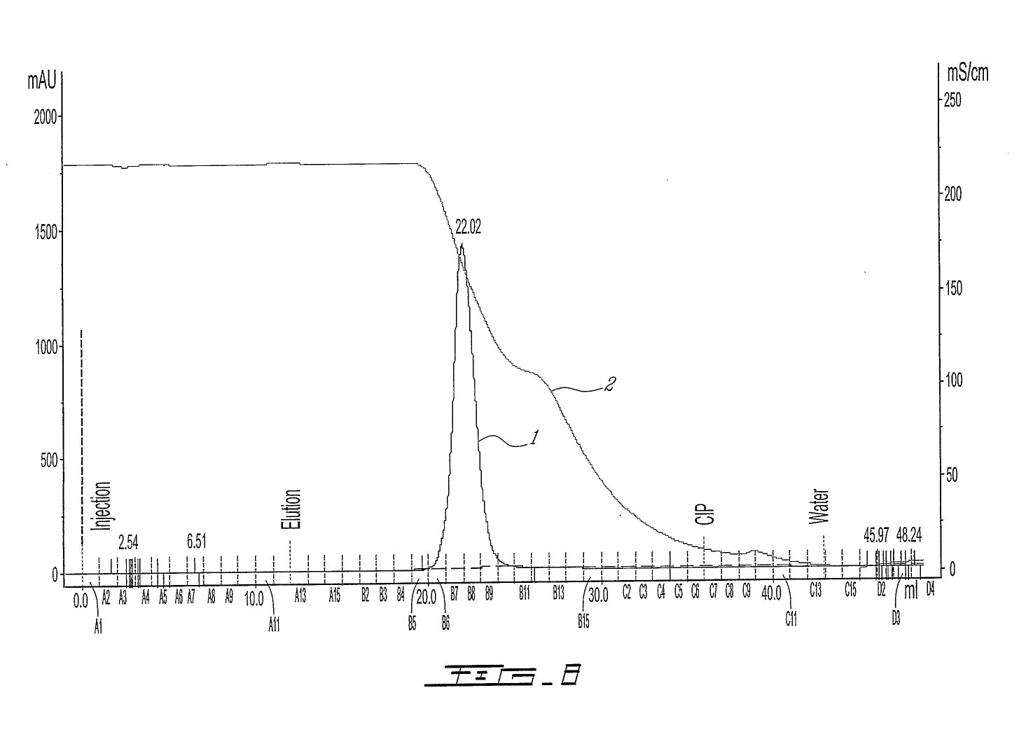

The resulting chromatogram (FIG. 7) shows that almost all of the applied protein passed through the resin without adsorption.

Selected fractions, analyzed for lactoferrin content by HPLC (Table 3), show only a small increase in lactoferrin purity in a representative pool of fractions from the main peak. There appears to be a systematic error of unknown origin in regard to lactoferrin recovery, having no material bearing on the degree of lactoferrin purification achieved.

TABLE 3
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC50-S1 | Feed | 16.09 | 1.0 | 16.09 | 94.08 | |
| 05CR1-HIC50-S3 | A2 | 2.10 | 1.0 | 2.10 | 96.10 | 13.05 |
| 05CR1-HIC50-S4 | A3 | 3.51 | 1.0 | 3.51 | 95.98 | 21.82 |
| 05CR1-HIC50-S5 | A4 | 0.37 | 1.0 | 0.37 | 95.64 | 2.32 |
| | | | | Total | | 37.19 |
| | | | | recovery: | | |
| A2-A4 Pool: | % purity: | 96.0 | % | 37.2 | | |
| | | | recovery: | | | |

### Example 6

### Bind-and-Elute Purification on Sephacryl™ S-400 (Run 05CR1-HIC51)

Partially purified bovine lactoferrin was dissolved and purified exactly as in Example 5 except that the equilibration buffer consisted of 20 mM sodium phosphate and 1.7 M ammonium sulfate, titrated with NaOH to pH 7.0.

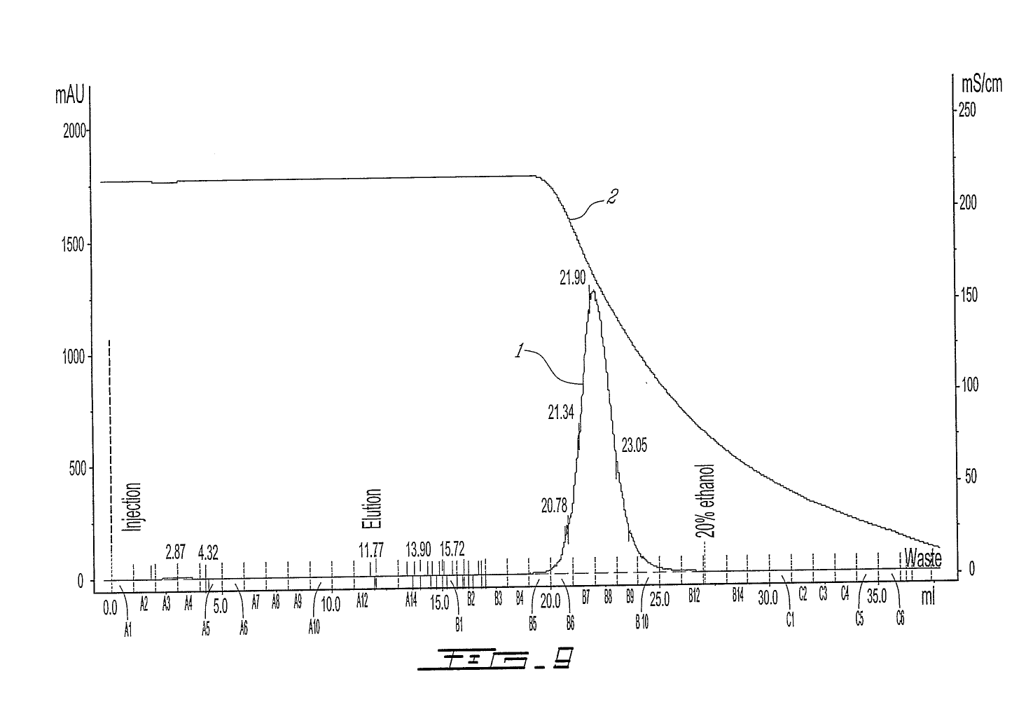

The resulting chromatogram (FIG. 8) shows that almost all of the applied protein adsorbed to the resin, and was eluted as the conductivity began to decrease from its initial value.

Selected fractions were analyzed for lactoferrin content by HPLC (Table 4).

TABLE 4
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC51-S1 | Feed | 7.72 | 1.0 | 7.72 | 94.64 | |
| 05CR1-HIC51-S6 | B6 | 0.05 | 1.0 | 0.05 | 94.09 | 0.62 |
| 05CR1-HIC51-S7 | B7 | 2.31 | 1.0 | 2.31 | 97.64 | 29.93 |
| 05CR1-HIC51-S8 | B8 | 3.32 | 1.0 | 3.32 | 98.83 | 43.04 |
| 05CR1-HIC51-S9 | B9 | 2.36 | 1.0 | 2.36 | 97.61 | 30.61 |
| 05CR1-HIC51-S10 | B10 | 0.41 | 1.0 | 0.41 | 91.55 | 5.28 |
| | | | | Total | | 109.47 |
| | | | | recovery: | | |
| B7-B9 Pool: | % purity: | 98.1 | % | 103.6 | | |
| | | | recovery: | | | |

The results show a substantial increase in lactoferrin purity to 98.1% in a representative pool of fractions from the main peak. The recovery of lactoferrin applied in the feed solution is somewhat uncertain but appears to be at least 95%.

### Example 7

### Purification of Euro Protein Lactoferrin on Sephacryl™ S-400 (Run 05CR1-HIC56)

Partially purified lactoferrin (Euro Proteins, Wapakoneta, Ohio, USA; “Europrot”, Lot No. EP2261-03; approximately 82 percent pure by HPLC) was dissolved and purified exactly as in Example 6.

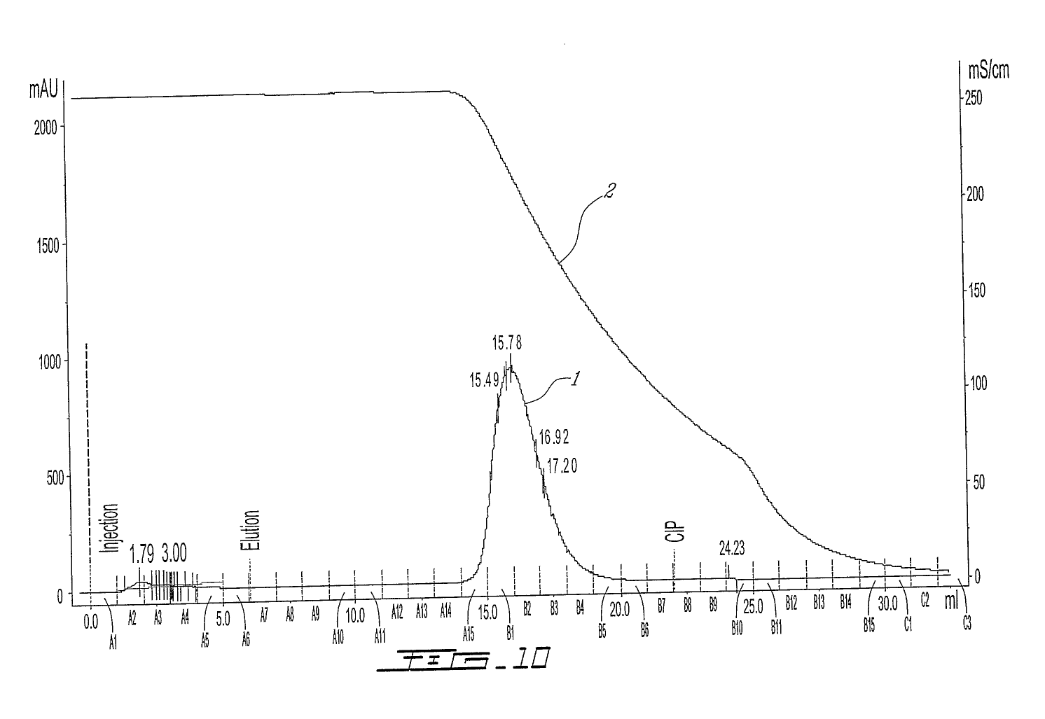

The resulting chromatogram (FIG. 9) shows that almost all of the applied protein adsorbed to the resin, and was eluted as the conductivity began to decrease from its initial value.

Selected fractions were analyzed for lactoferrin content by HPLC (Table 5).

TABLE 5
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC56-S1 | Feed | 7.45 | 1 | 7.45 | 86.90 | |
| 05CR1-HIC56-S5 | B6 | 0.09 | 1 | 0.09 | 87.91 | 1.27 |
| 05CR1-HIC56-S6 | B7 | 1.12 | 1 | 1.12 | 90.00 | 15.00 |
| 05CR1-HIC56-S7 | B8 | 3.69 | 1 | 3.69 | 92.08 | 49.57 |
| 05CR1-HIC56-S8 | B9 | 2.17 | 1 | 2.17 | 86.24 | 29.20 |
| 05CR1-HIC56-S9 | B10 | 0.49 | 1 | 0.49 | 67.94 | 6.55 |
| | | | | Total | | 101.58 |
| | | | | recovery: | | |
| B7-B8 Pool: | % purity: | 91.6 | % | 64.6 | | |
| | | | recovery: | | | |

The results show that the lactoferrin feed solution was somewhat increased in purity relative to the starting material, presumably due to the insolubility of one or more contaminants at the high concentration of ammonium sulfate employed. More importantly, the results show an increase in lactoferrin purity to 91.6% in a representative pool of fractions from the main peak, with an apparent recovery of 64.6% of the lactoferrin applied in the feed solution.

### Example 8

### Bind-and-Elute Purification on SP Sepharose™ (Run 05CR1-HIC55)

Partially purified lactoferrin was dissolved and purified as in Example 3 with the following three exceptions: the equilibration buffer consisted of 20 mM sodium phosphate and 2.3 M ammonium sulfate, titrated with NaOH to pH 7.0; the chromatographic resin used was SP Sepharose Fast Flow (GE Healthcare); and lactoferrin feed solution was clarified using a Millex® HV, 0.45-μm pore size, membrane filter (Millipore).

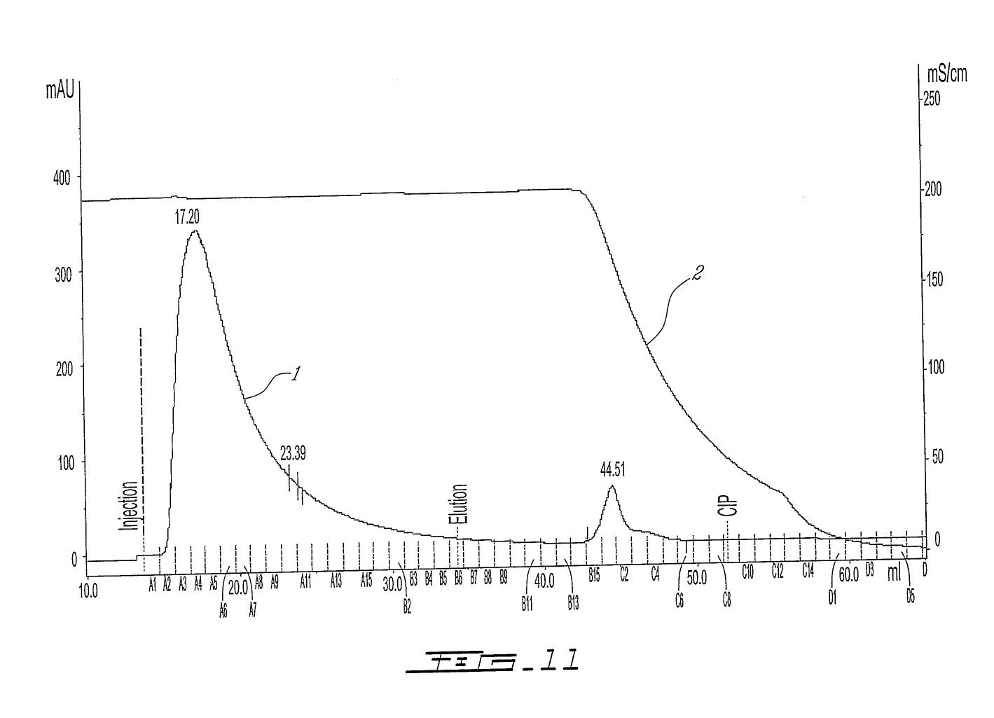

The resulting chromatogram (FIG. 10) shows that most of the applied protein adsorbed to the resin, and was eluted as the conductivity began to decrease from its initial value.

Selected fractions were analyzed for lactoferrin content by HPLC (Table 6).

TABLE 6
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC55-S1 | Feed | 7.18 | 1 | 7.18 | 95.22 | |
| 05CR1-HIC55-S2 | A2 | 0.00 | 1 | 0.00 | 74.33 | 0.04 |
| 05CR1-HIC55-S3 | A3 | 0.01 | 1 | 0.01 | 46.82 | 0.12 |
| 05CR1-HIC55-S4 | A4 | 0.01 | 1 | 0.01 | 19.65 | 0.13 |
| 05CR1-HIC55-S7 | A15 | 0.04 | 1 | 0.04 | 95.19 | 0.50 |
| 05CR1-HIC55-S8 | B1 | 0.95 | 1 | 0.95 | 98.24 | 13.18 |
| 05CR1-HIC55-S9 | B2 | 2.73 | 1 | 2.73 | 99.73 | 37.97 |
| 05CR1-HIC55-S10 | B3 | 2.24 | 1 | 2.24 | 98.74 | 31.16 |
| 05CR1-HIC55-S11 | B4 | 0.94 | 1 | 0.94 | 97.39 | 13.08 |
| | | | | Total | | 96.18 |
| | | | | recovery: | | |
| B1-B4 Pool: | % purity: | 98.9 | % | 95.4 | | |
| | | | recovery: | | | |

The results show a very substantial increase in lactoferrin purity to 98.9% in a representative pool of fractions from the main peak, and with 95.4% recovery of the lactoferrin applied in the feed solution.

### Example 9

### Flowthrough Purification on Toyopearl® Ether (Run 05CR1-HIC28)

Partially purified lactoferrin was dissolved and purified as in Example 3 except that the chromatographic resin used was Toyopearl® Ether 650M (Tosoh Bioscience).

The resulting chromatogram (FIG. 11) shows that most of the applied protein passed through the resin, although evidently reversibly adsorbing to the resin in the process.

Selected fractions were analyzed for lactoferrin content by HPLC (Table 7).

TABLE 7
Lactoferrin purity in chromatographic fractions

| | | | Volume | | | |
|---|---|---|---|---|---|---|
| | | | of | Total | | |
| | | Lactoferrin | fraction | Lactoferrin | % purity | % |
| Sample name | Description | mg/ml | ml (g) | mg | by area | recovery |
| 05CR1-HIC28-S1 | feed | 9.27 | 1.00 | 9.27 | 92.87 | |
| 05CR1-HIC28-S2 | A2 | 0.00 | 1.00 | 0.00 | 63.61 | 0.05 |
| 05CR1-HIC28-S3 | A3 | 0.30 | 1.00 | 0.30 | 80.09 | 3.22 |
| 05CR1-HIC28-S4 | A4 | 1.32 | 1.00 | 1.32 | 93.96 | 14.27 |
| 05CR1-HIC28-S5 | A5 | 1.16 | 1.00 | 1.16 | 97.26 | 12.52 |
| 05CR1-HIC28-S6 | A6 | 1.66 | 1.00 | 1.66 | 98.34 | 17.91 |
| 05CR1-HIC28-S7 | A7 | 0.88 | 1.00 | 0.88 | 99.07 | 9.53 |
| 05CR1-HIC28-S8 | A8 | 0.68 | 1.00 | 0.68 | 99.45 | 7.35 |
| 05CR1-HIC28-S9 | A9 | 0.51 | 1.00 | 0.51 | 99.69 | 5.53 |
| 05CR1-HIC28- | A10 | 0.43 | 1.00 | 0.43 | 99.74 | 4.66 |
| S10 | | | | | | |
| 05CR1-HIC28- | A11 | 0.31 | 1.00 | 0.31 | 99.92 | 3.32 |
| S11 | | | | | | |
| 05CR1-HIC28- | A12 | 0.25 | 1.00 | 0.25 | 99.92 | 2.64 |
| S12 | | | | | | |
| 05CR1-HIC28- | A13 | 0.20 | 1.00 | 0.20 | 99.58 | 2.13 |
| S13 | | | | | | |
| 05CR1-HIC28- | A14 | 0.16 | 1.00 | 0.16 | 99.52 | 1.67 |
| S14 | | | | | | |
| 05CR1-HIC28- | B15 | 0.02 | 1.00 | 0.02 | 97.04 | 0.17 |
| S16 | | | | | | |
| 05CR1-HIC28- | C1 | 0.10 | 1.00 | 0.10 | 99.87 | 1.12 |
| S17 | | | | | | |
| 05CR1-HIC28- | C2 | 0.09 | 1.00 | 0.09 | 99.38 | 0.94 |
| S18 | | | | | | |
| 05CR1-HIC28- | C3 | 0.07 | 1.00 | 0.07 | 99.60 | 0.78 |
| S19 | | | | | | |
| | | | | Total | | 87.83 |
| | | | | recovery: | | |
| A5-C3 Pool: | % purity: | 98.8 | % | 70.3 | | |
| | | | recovery: | | | |

The results show that, following the initial few fractions in the flowthrough, all of the remaining fractions in the flowthrough and elution peak contain highly pure lactoferrin. Thus a representative pool of the latter had a purity of 98.8%, but with only 70.3% recovery of the lactoferrin applied in the feed solution.

### Example 10

### Bind-and-Elute Purification on Toyopearl® Ether (Run 05CR1-HIC37)

Partially purified lactoferrin was dissolved and purified exactly as in Example 9 except that the equilibration buffer consisted of 20 mM sodium phosphate and 1.8 M ammonium sulfate, titrated with NaOH to pH 7.0, and lactoferrin feed solution was clarified using a Millex® HV, 0.45-μm pore size, membrane filter (Millipore).

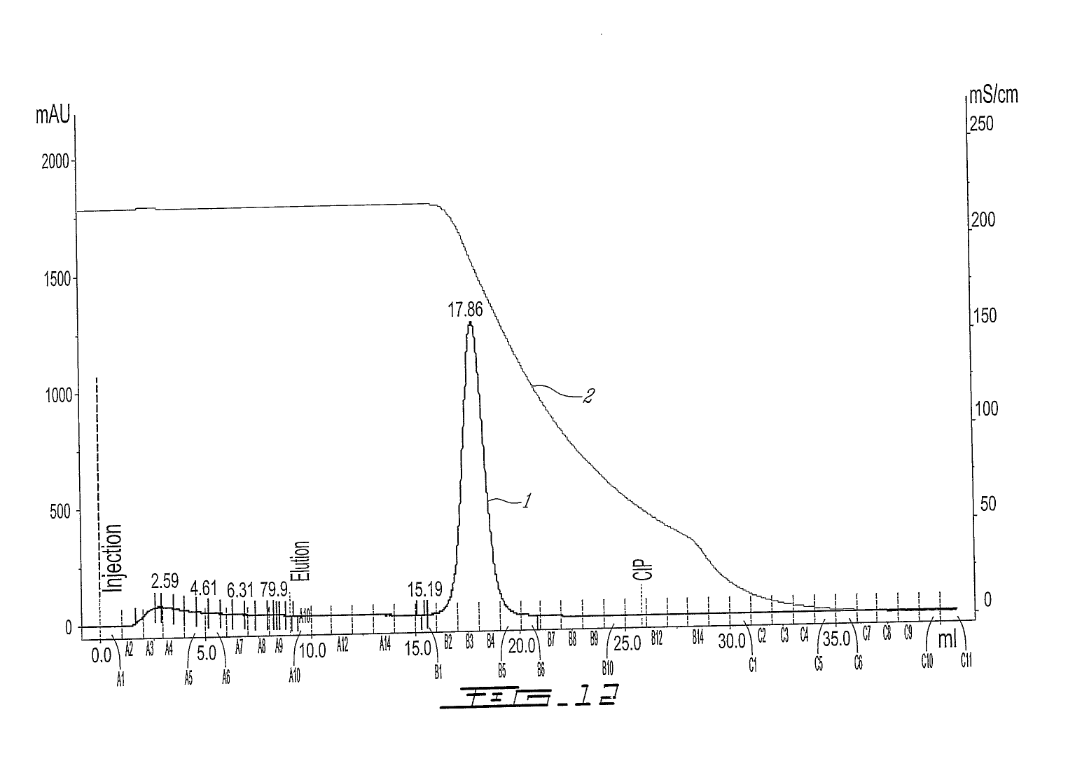

The resulting chromatogram (FIG. 12) shows that most of the applied protein adsorbed to the resin, and was eluted as the conductivity began to decrease from its initial value.

Selected fractions were analyzed for lactoferrin content by HPLC (Table 8).

TABLE 8
Lactoferrin purity in chromatographic fractions

| | | | Volume of | Total | % | |
|---|---|---|---|---|---|---|
| | | Lactoferrin | fraction | Lactoferrin | purity | % |
| Sample name | Description | mg/ml | ml (g) | mg | by area | recovery |
| 05CR1-HIC37-S1 | feed | 13.56 | 1.0 | 13.56 | 94.20 | |
| 05CR1-HIC37-S2 | A2 | 0.00 | 1.0 | 0.00 | 77.98 | 0.03 |
| 05CR1-HIC37-S3 | A3 | 0.06 | 1.0 | 0.06 | 68.03 | 0.48 |
| 05CR1-HIC37-S4 | A4 | 0.18 | 1.0 | 0.18 | 84.06 | 1.29 |
| 05CR1-HIC37-S5 | A5 | 0.19 | 1.0 | 0.19 | 92.16 | 1.36 |
| 05CR1-HIC37-S6 | A6 | 0.05 | 1.0 | 0.05 | 94.05 | 0.38 |
| 05CR1-HIC37-S10 | B2 | 0.12 | 1.0 | 0.12 | 98.35 | 0.90 |
| 05CR1-HIC37-S11 | B3 | 1.43 | 1.0 | 1.43 | 99.43 | 10.54 |
| 05CR1-HIC37-S12 | B4 | 7.21 | 1.0 | 7.21 | 97.50 | 53.18 |
| 05CR1-HIC37-S13 | B5 | 1.30 | 1.0 | 1.30 | 92.47 | 9.61 |
| 05CR1-HIC37-S14 | B6 | 0.20 | 1.0 | 0.20 | 80.31 | 1.48 |
| | | | | Total | | 79.24 |
| | | | | recovery: | | |
| B2-B4 Pool: | % purity: | 97.8 | % recovery: | 64.6 | | |

The results show an increase in lactoferrin purity to 97.8% in a representative pool of fractions from the main peak, but with apparently only 64.6% recovery of the lactoferrin applied in the feed solution.

### Example 11

### Bind-and-Elute Purification on Toyopearl® Ether (Run 05CR1-HIC29)

Partially purified lactoferrin was dissolved and purified exactly as in Example 9 except that the equilibration buffer consisted of 20 mM sodium phosphate and 2.0 M ammonium sulfate, titrated with NaOH to pH 7.0, and lactoferrin feed solution was clarified using a Millex® GV, 0.22-μm pore size, membrane filter (Millipore).

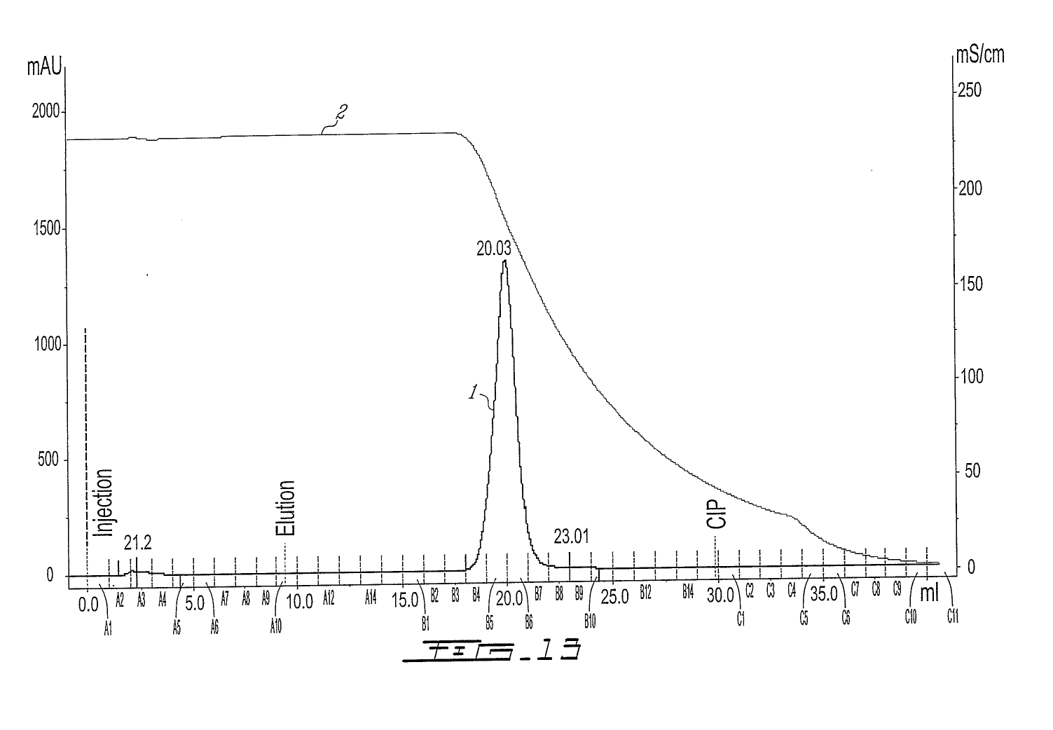

The resulting chromatogram (FIG. 13) shows that almost all of the applied protein adsorbed to the resin, and was eluted as the conductivity began to decrease from its initial value.

Selected fractions were analyzed for lactoferrin content by HPLC (Table 9).

TABLE 9
Lactoferrin purity in chromatographic fractions

| | | | Volume of | Total | % | |
|---|---|---|---|---|---|---|
| | | Lactoferrin | fraction | Lactoferrin | purity | % |
| Sample name | Description | mg/ml | ml (g) | mg | by area | recovery |
| 05CR1-HIC29-S1 | Feed | 5.91 | 1.0 | 5.91 | 96.06 | |
| 05CR1-HIC29-S3 | A2′ | 0.00 | 1.0 | 0.00 | 74.31 | 0.03 |
| 05CR1-HIC29-S4 | A3′ | 0.00 | 1.0 | 0.00 | 7.02 | 0.04 |
| 05CR1-HIC29-S10 | B3′ | 0.00 | 1.0 | 0.00 | 92.88 | 0.07 |
| 05CR1-HIC29-S11 | B4′ | 0.08 | 1.0 | 0.08 | 97.87 | 1.35 |
| 05CR1-HIC29-S12 | B5′ | 2.12 | 1.0 | 2.12 | 98.83 | 35.79 |
| 05CR1-HIC29-S13 | B6′ | 1.90 | 1.0 | 1.90 | 97.83 | 32.23 |
| 05CR1-HIC29-S14 | B7′ | 1.28 | 1.0 | 1.28 | 94.87 | 21.69 |
| | | | | Total | | 80.80 |
| | | | | recovery: | | |
| B4′-B7′ Pool: | % purity: | 97.5 | % recovery: | 91.0 | | |

The results show an increase in lactoferrin purity to 97.5% in a representative pool of fractions from the main peak, and with 91.0% recovery of the lactoferrin applied in the feed solution.

Note that in Table 9, the fractions are designated with a prime for the reason explained under Example 3.

### Example 12

### Bind-and-Elute Purification on Phenyl Sepharose HP (Run 05CR1-HIC40)

Partially purified lactoferrin was dissolved and purified as in Example 3 with the following exceptions: the equilibration buffer consisted of 20 mM sodium phosphate and 1.4 M ammonium sulfate, titrated with NaOH to pH 7.0, as well as 30 mg/L of Tween™ 20 (Sigma-Aldrich); the elution buffer consisted of 20 mM sodium phosphate, titrated with NaOH to pH 7.0, and 30 mg/L of Tween™ 20; the chromatographic resin used was Phenyl Sepharose™ HP (GE Healthcare); and lactoferrin feed solution was clarified using a Millex® HV, 0.45-μm pore size, membrane filter (Millipore).

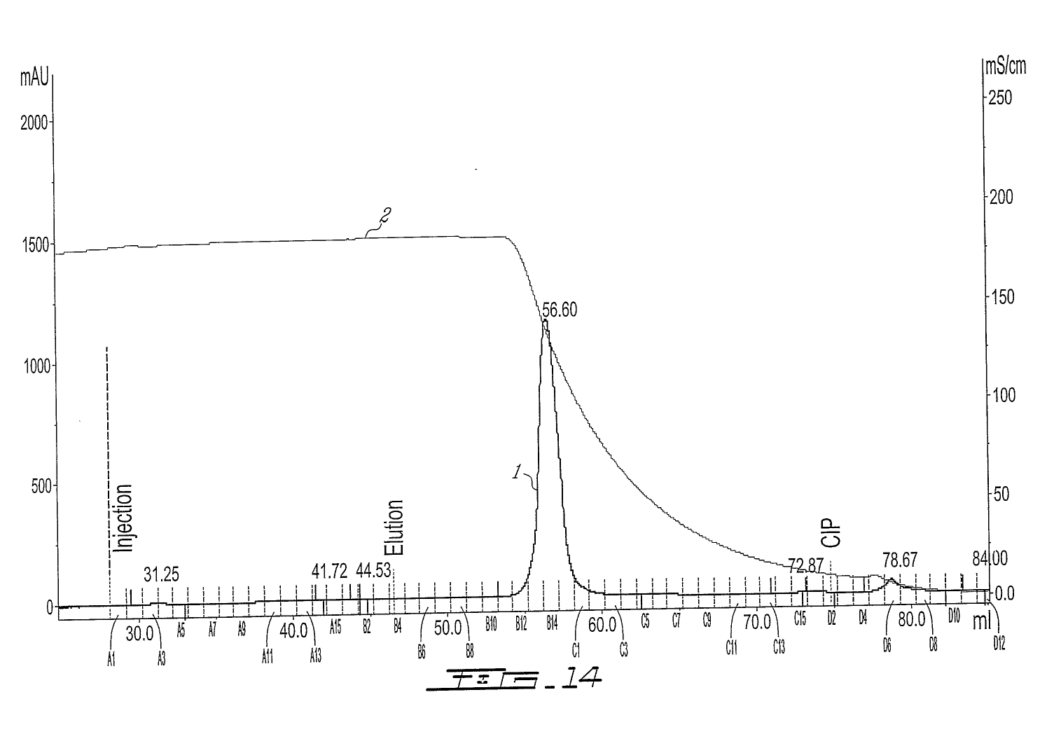

The resulting chromatogram (FIG. 14) shows that almost all of the applied protein adsorbed to the resin, and was eluted as the conductivity began to decrease from its initial value.

Selected fractions were analyzed for lactoferrin content by HPLC (Table 10).

TABLE 10
Lactoferrin purity in chromatographic fractions

| | | | Volume of | Total | % | |
|---|---|---|---|---|---|---|
| | | Lactoferrin | fraction | Lactoferrin | purity | % |
| Sample name | Description | mg/ml | ml (g) | mg | by area | recovery |
| 05CR1-HIC40-S1 | feed | 7.60 | 1.0 | 7.60 | 92.91 | |
| 05CR1-HIC40-S3 | A3 | 0.00 | 1.0 | 0.00 | 13.47 | 0.03 |
| 05CR1-HIC40-S4 | A4 | 0.00 | 1.0 | 0.00 | 2.63 | 0.03 |
| 05CR1-HIC40-S9 | A9 | 0.01 | 1.0 | 0.01 | 86.04 | 0.15 |
| 05CR1-HIC40-S12 | A12 | 0.02 | 1.0 | 0.02 | 92.06 | 0.33 |
| 05CR1-HIC40-S16 | B1 | 0.03 | 1.0 | 0.03 | 90.25 | 0.44 |
| 05CR1-HIC40-S27 | B12 | 0.06 | 1.0 | 0.06 | 96.39 | 0.84 |
| 05CR1-HIC40-S28 | B13 | 0.46 | 1.0 | 0.46 | 97.81 | 6.11 |
| 05CR1-HIC40-S29 | B14 | 2.70 | 1.0 | 2.70 | 98.54 | 35.51 |
| 05CR1-HIC40-S30 | B15 | 2.90 | 1.0 | 2.90 | 98.98 | 38.11 |
| 05CR1-HIC40-S31 | C1 | 0.71 | 1.0 | 0.71 | 93.20 | 9.40 |
| 05CR1-HIC40-S32 | C2 | 0.16 | 1.0 | 0.16 | 83.58 | 2.17 |
| 05CR1-HIC40-S33 | C3 | 0.06 | 1.0 | 0.06 | 80.41 | 0.73 |
| 05CR1-HIC40-S36 | C6 | 0.02 | 1.0 | 0.02 | 70.17 | 0.25 |
| 05CR1-HIC40-S41 | C11 | 0.01 | 1.0 | 0.01 | 27.60 | 0.08 |
| 05CR1-HIC40-S45 | C15 | 0.00 | 1.0 | 0.00 | 15.66 | 0.05 |
| 05CR1-HIC40-S46 | CIP Pool | 0.00 | 4.0 | 0.01 | 1.80 | 0.12 |
| | | | | Total | | 94.34 |
| | | | | recovery: | | |
| B13-B15 Pool: | % purity: | 98.7 | % recovery: | 79.7 | | |

The results show a substantial increase in lactoferrin purity to 98.7% in a representative pool of fractions from the main peak, and with 79.7% recovery of the lactoferrin applied in the feed solution.

### Example 13

### Bind-and-Elute Purification on Phenyl Sepharose™ HP (Run 05CR1-HIC27)

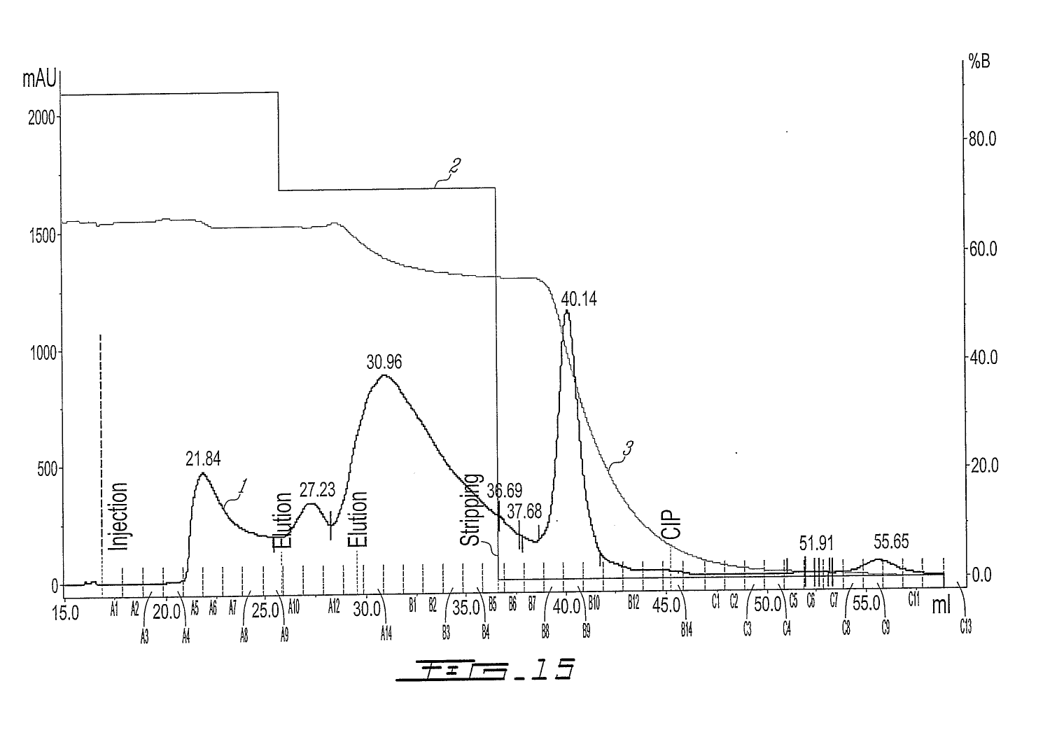

Partially purified lactoferrin was dissolved and purified as in Example 12 with the following exceptions:
1. The lactoferrin dissolution buffer and column equilibration buffer consisted of a 90:10 v/v mixture of 20 mM sodium phosphate and 1.65 M ammonium sulfate, titrated with NaOH to pH 7.0 (Buffer B), with 20 mM sodium phosphate, titrated with NaOH to pH 7.0, and 550 mg/L of Tween™ 20 (Buffer A);
2. The lactoferrin feed solution was prepared at a nominal concentration of 20 mg/ml, and 2.2 ml of this solution was loaded onto the column;
3. The lactoferrin feed solution was clarified by filtration through a Pall Acrodisce Supor® 0.22-μm pore size membrane filter;
4. The flow rate up to the point marked “Stripping” on the chromatogram (FIG. 15) was 0.15 ml/min, and thereafter 1.0 ml/min;
5. At the end of fraction A9, elution with a 72:28 v/v mixture of Buffer B and Buffer A was begun.

The resulting chromatogram (FIG. 15) shows that substantial material exited the column starting with the flowthrough and continuing to the stripping peak, but predominantly during elution with 72% v/v Buffer B.

Selected fractions were analyzed for lactoferrin content by HPLC (Table 11).

TABLE 11
Lactoferrin purity in chromatographic fractions

| | | | Volume | | | |
|---|---|---|---|---|---|---|
| | | | of | Total | % | |
| | | Lactoferrin | fraction | Lactoferrin | purity | % |
| Sample name | Description | mg/ml | ml (g) | mg | by area | recovery |
| 05CR1-HIC27-S1 | feed | 18.29 | 2.2 | 40.24 | 94.70 | |
| 05CR1-HIC27-S5 | A5 | 0.09 | 1.0 | 0.09 | 63.71 | 0.48 |
| 05CR1-HIC27-S7 | A7 | 1.20 | 1.0 | 1.20 | 88.99 | 6.54 |
| 05CR1-HIC27-S9 | A9 | 0.77 | 1.0 | 0.77 | 97.00 | 4.19 |
| 05CR1-HIC27-S11 | A11 | 0.96 | 1.0 | 0.96 | 98.92 | 5.25 |
| 05CR1-HIC27S-13 | A13 | 1.26 | 1.0 | 1.26 | 99.21 | 6.88 |
| 05CR1-HIC27-S14 | A14 | 2.12 | 1.0 | 2.12 | 98.99 | 11.56 |
| 05CR1-HIC27-S16 | B1 | 3.15 | 1.0 | 3.15 | 98.03 | 17.20 |
| 05CR1-HIC27-S18 | B3 | 2.58 | 1.0 | 2.58 | 98.31 | 14.11 |
| 05CR1-HIC27-S20 | B5 | 2.00 | 1.0 | 2.00 | 98.35 | 10.92 |
| 05CR1-HIC27-S22 | B7 | 0.85 | 1.0 | 0.85 | 95.92 | 4.66 |
| 05CR1-HIC27-S24 | B9 | 2.66 | 1.0 | 2.66 | 94.33 | 14.52 |
| 05CR1-HIC27-S26 | B11 | 0.59 | 1.0 | 0.59 | 80.32 | 3.25 |
| 05CR1-HIC27-S31 | CIP pool | 0.02 | 3.0 | 0.05 | 9.50 | 0.25 |
| | | | | Total | | 45.36 |
| | | | | recovery: | | |
| A9-B5 Pool: | % purity: | 98.4 | % | 31.9 | | |
| | | | recovery: | | | |

The results show a substantial increase in lactoferrin purity to 98.4% in a representative pool of fractions combining the tail of the flowthrough peak with the 72% v/v Buffer B peak. Since only alternate fractions were analyzed, the recovery in this pool of the lactoferrin applied in the feed solution can only be approximately estimated at about 65%.

### Example 14

### Bind-and-Elute Purification on Phenyl Sepharose™ HP (Run 3.4-L Phenyl Sepharose R1)

Partially purified lactoferrin (DMV International; Lot No. 10191343; approximately 92 percent pure by HPLC) was dissolved at a concentration of 20 mg/ml in freshly prepared 20 mM sodium phosphate buffer, pH 7.0, containing 1.485 M ammonium sulphate and 0.005% v/v Tween® 20. The apparently colloidal material present was removed by filtration through a combination graded depth-membrane filter (Millipore Opticap®, 0.5/0.2/0.22-μm pore size) followed by an absolute membrane filter (Millipore Millipak®, 0.22-μm pore size).

The solution was applied at a superficial velocity of 100 cm/h, and a loading of 43 mg per ml of adsorbent, to a chromatography column containing an 11-cm bed of Phenyl Sepharose HP resin previously equilibrated with the above buffer.

The column was washed with buffer as above. A small flowthrough peak emerged, then after approximately 1.5-2 bed volumes a front containing a substantial amount of lower molecular-weight impurities as well as some lactoferrin.

After the maximum concentration of this peak had exited the column, fraction collection was begun, since the tail of this peak contains highly pure lactoferrin.

Concomitantly with the start of fraction collection, an eluent solution of 20 mM sodium phosphate buffer, pH 7.0, containing 1.2 M ammonium sulphate and 0.014% v/v Tween 20 was applied to the column at the same superficial velocity. This caused a peak to elute which contains highly pure lactoferrin in a total of approximately 2.0-2.5 column volumes, followed by a long tail containing both higher and lower molecular-weight impurities mixed with some lactoferrin.

Part-way through this tail, the column was stripped of strongly adherent substances, and simultaneously regenerated, by eluting with 20 mM sodium phosphate buffer, pH 7.0, containing 0.05% v/v Tween 20.

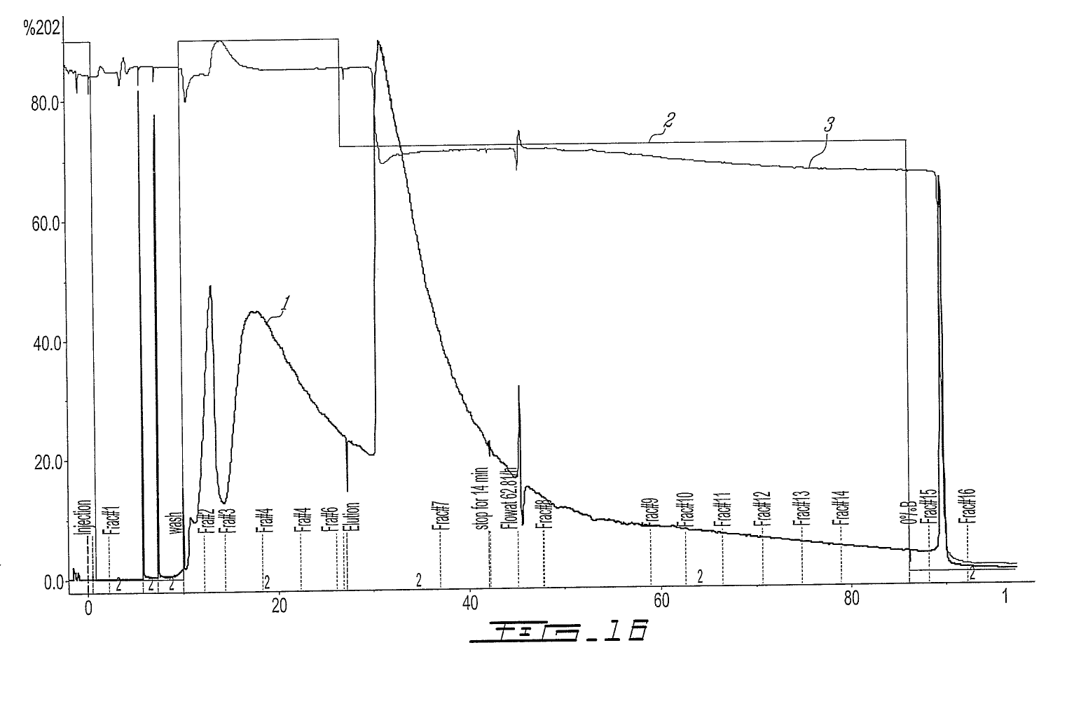

The resulting chromatogram is shown in FIG. 16.

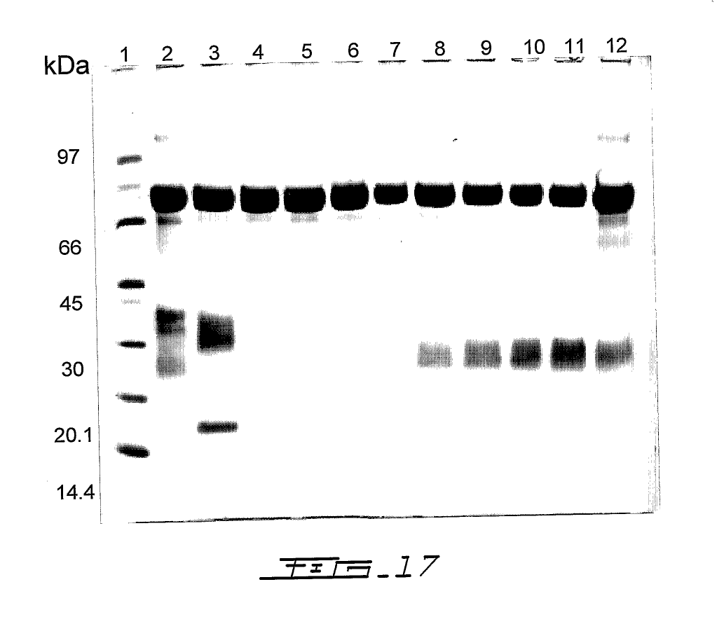

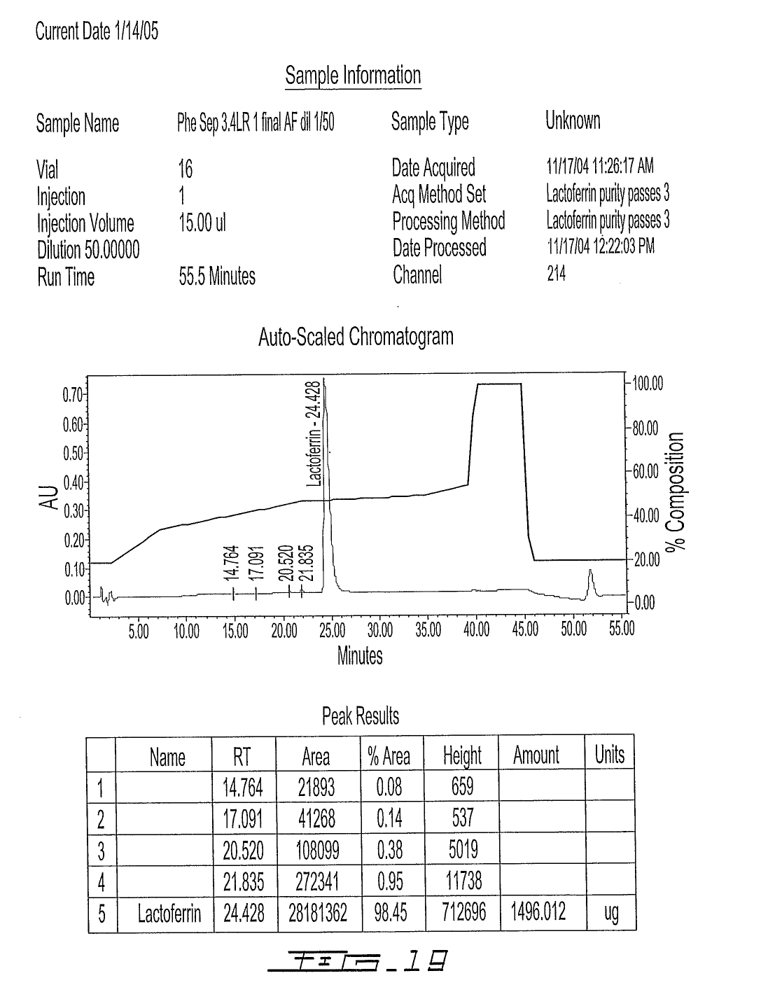

The combined fractions 1 to 5 contained approximately 75 percent of the lactoferrin starting material, and had a purity of approximately 98 percent by HPLC (FIGS. 17, 18 and 19).

FIG. 17 illustrates a SDS-PAGE gel electrophoresis of bovine lactoferrin (20 mg/ml) purified on Phenyl Sepharose™ HP resin in accordance with this process. Gel wells were loaded accordingly as follows:

| Product loaded | Lanes |
|---|---|
| LMW Pharmacia marker | 1 |
| Feed 20 mg/ml after filtration diluted 1/40 (≈6.5 μg loaded) | 2 |
| Flow through | 3 |
| Eluate Fraction #1 | 4 |
| Eluate Fraction #2 | 5 |
| Eluate Fraction #3 | 6 |
| Eluate Fraction #4 | 7 |
| Eluate Fraction #5 | 8 |
| Eluate Fraction #6 | 9 |
| Eluate Fraction #7 diafiltered | 10 |
| Eluate Fraction #8 diafiltered* | 11 |
| Eluate Fraction #9. | 12 |

The samples are diluted or concentrated to give the same O.D.₂₈₀ as the feed.
*Diafiltration against: 20 mM Na₂HPO₄ pH 7.0

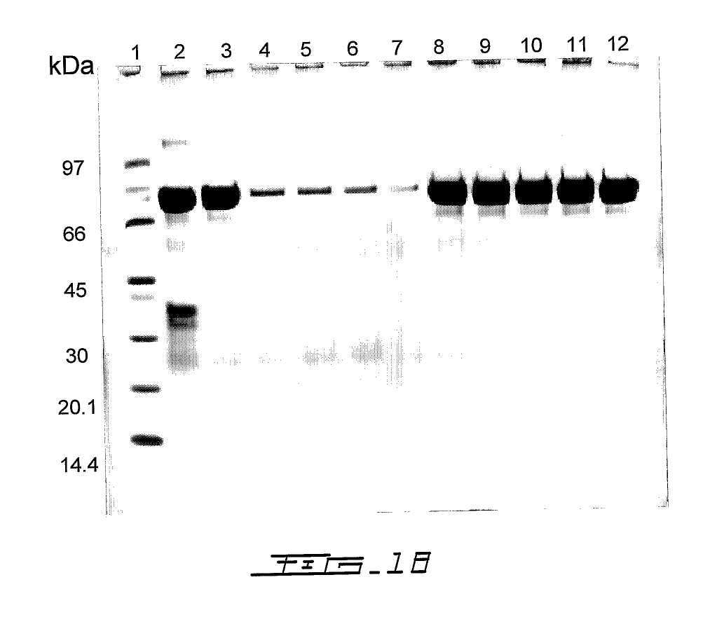

FIG. 18 illustrates a SDS-PAGE gel electrophoresis of bovine lactoferrin (20 mg/ml) purified on Phenyl Sepharose™ HP resin. Gel wells were loaded accordingly as follows:

| Product loaded | Lanes |
|---|---|
| LMW Pharmacia marker | 1 |
| Feed 20 mg/ml after filtration diluted 1/40 (≈6.5 μg loaded) | 2 |
| Pool eluate fractions 1 to 5 inclusively | 3 |
| Permeate of concentration step bottle #1 diafiltered * | 4 |
| Permeate of concentration step bottle #2 diafiltered | 5 |
| Permeate of concentration step bottle #3 diafiltered | 6 |
| Permeate of diafiltration | 7 |
| Final product before filtration | 8 |
| Final product after 4.5 μm | 9 |
| Final product after 1.2/0.5 μm | 10 |
| Final product | 11 |
| Final product | 12 |

The samples are diluted or concentrated to give the same O.D.₂₈₀ as the feed (except the permeates).
* diafiltration against: 20 mM Na₂HPO₄ pH 7.0 needed.

### Example 15

### Bind-and-Elute Purification on Phenyl Sepharose™ HP (Run 05CR1-HIC45)

Partially purified lactoferrin was dissolved and purified as in Example 12 with the following exceptions:
a) The lactoferrin dissolution buffer and column equilibration buffer consisted of 0.2 M sodium phosphate, 0.2 M acetic acid and 2.0 M sodium chloride, titrated with NaOH to pH 4.0;
b) After loading, the column was washed with equilibration buffer until the absorbance returned to baseline, then washed with a solution consisting of 0.2 M sodium phosphate, 0.2 M acetic acid and 0.5 M sodium chloride, titrated with NaOH to pH 4.0;
c) The resin was eluted with a solution consisting of 0.2 M sodium phosphate and 0.2 M acetic acid, titrated with NaOH to pH 5.0.

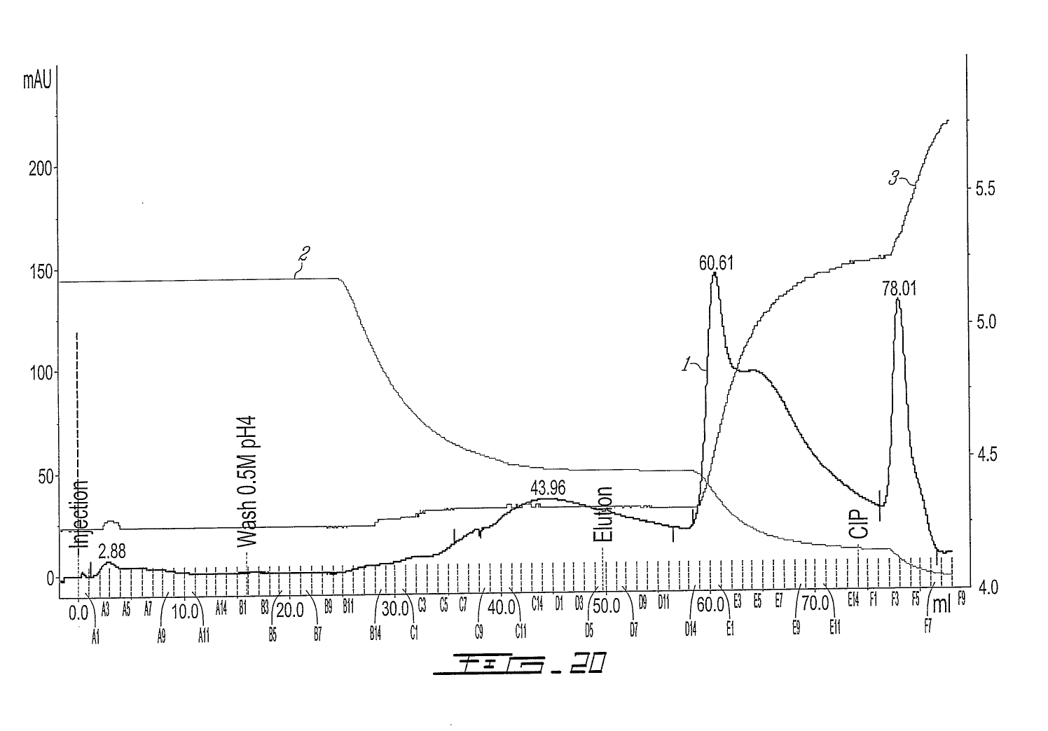

The resulting chromatogram (FIG. 20) shows that substantial material exited the column starting with the wash peak and continuing to the CIP (cleaning-in-place) peak.

Selected fractions were analyzed for lactoferrin content by HPLC (Table 12).

TABLE 12
Lactoferrin purity in chromatographic fractions.

| | | | Volume of | Total | % | |
|---|---|---|---|---|---|---|
| | | Lactoferrin | fraction | Lactoferrin | purity | % |
| Sample name | Description | mg/ml | ml (g) | mg | by area | recovery |
| 05CR1-HIC45-S1 | Feed | 7.43 | 1.0 | 7.43 | 92.31 | |
| 05CR1-HIC45-S2 | A3 + A4 | 0.00 | 2.0 | 0.00 | 0.00 | 0.00 |
| 05CR1-HIC45-S3 | C1-C5 | 0.01 | 5.0 | 0.03 | 65.98 | 0.43 |
| 05CR1-HIC45-S4 | C6-C10 | 0.02 | 5.0 | 0.12 | 61.32 | 1.68 |
| 05CR1-HIC45-S5 | C11-C15 | 0.09 | 5.0 | 0.47 | 98.87 | 6.31 |
| 05CR1-HIC45-S6 | D1-D6 | 0.10 | 6.0 | 0.60 | 99.22 | 8.11 |
| 05CR1-HIC45-S8 | D14 | 0.06 | 1.0 | 0.06 | 99.82 | 0.76 |
| 05CR1-HIC45-S9 | D15 | 0.11 | 1.0 | 0.11 | 100.00 | 1.42 |
| 05CR1-HIC45-S10 | E1 | 0.30 | 1.0 | 0.30 | 99.98 | 4.04 |
| 05CR1-HIC45-S11 | E2 | 0.47 | 1.0 | 0.47 | 100.00 | 6.32 |
| 05CR1-HIC45-S12 | E3 | 0.37 | 1.0 | 0.37 | 98.25 | 4.99 |
| 05CR1-HIC45-S13 | E4 | 0.34 | 1.0 | 0.34 | 97.90 | 4.64 |
| 05CR1-HIC45-S15 | E6 | 0.32 | 1.0 | 0.32 | 98.34 | 4.25 |
| 05CR1-HIC45-S17 | E8 | 0.26 | 1.0 | 0.26 | 98.29 | 3.48 |
| 05CR1-HIC45-S19 | E10 | 0.21 | 1.0 | 0.21 | 98.43 | 2.79 |
| 05CR1-HIC45-S20 | CIP Pool | 0.18 | 5.0 | 0.88 | 81.65 | 11.78 |
| | | | | Total | | 61.01 |
| | | | | recovery: | | |
| C11-E10 Pool: | % purity: | 98.9 | % | 47.1 | | |
| | | | recovery: | | | |

The results show a very substantial increase in lactoferrin purity to 98.9% in a representative pool of fractions combining most of the wash peak with the elution peak, but with an apparent recovery of only 47.1% of the lactoferrin applied in the feed solution.

### Example 16

### Bind-and-Elute Purification on Phenyl Sepharose™ HP

Partially purified bovine lactoferrin (DMV International; approximately 92 percent pure by HPLC) was dissolved at a concentration of 10 mg/ml in 0.2 M acetic acid and 0.2 M sodium phosphate buffer, pH 4.0, containing 2.0 M sodium chloride (final pH about 3.8). The apparently colloidal material present was removed by filtration through a membrane filter (Millipore Millex®GV, 0.22-μm pore size).

The lactoferrin solution was applied at a superficial velocity of 150 cm/h, and a loading of 10 mg per ml of adsorbent, to a 5-cm bed of Phenyl Sepharose™ HP resin previously equilibrated with the above buffer.

The column was washed with buffer as above until the absorbance returned to baseline, then washed with 0.2 M acetic acid and 0.2 M sodium phosphate buffer, pH 4.0, containing 0.5 M sodium chloride, to remove impurities.

Purified lactoferrin was eluted with 0.2 M acetic acid and 0.2 M sodium phosphate buffer, pH 5.0, using the same flow rate as above. Fractions showing UV absorbance were analysed by SDS-PAGE using silver staining, showing substantial removal of both lower and higher molecular weight impurities in the peak that eluted at pH 5 (Fraction 22 to Fraction 30; FIG. 21).

FIG. 21 illustrates a SDS-PAGE gel electrophoresis of bovine lactoferrin (10 mg/ml) purified on Phenyl Sepharose™ HP resin. Gel wells were loaded accordingly as follows:

| Product loaded | Lanes |
|---|---|
| LMW Pharmacia marker | 1 |
| Feed 10 mg/ml diafiltered * diluted 1/20 (≈6.5 μg loaded) | 2 |
| Flow Through diafiltered | 3 |
| Wash Fr. 8-9-10 | 4 |
| Fraction 22 | 5 |
| Fraction 23 | 6 |
| Fraction 23 pH adjusted ** | 7 |
| Fraction 25 | 8 |
| Fraction 27 | 9 |
| Fraction 29 | 10 |
| Fraction 30 | 11 |
| Stripping with 80% isopropanol | 12 |

* Diafiltration against: 20 mM Na₂HPO₄ pH 7.0
** Once the sample was in sample buffer, NaOH was added to turn the color from green to purple.

### Example 17

### Lactoferrin Stability

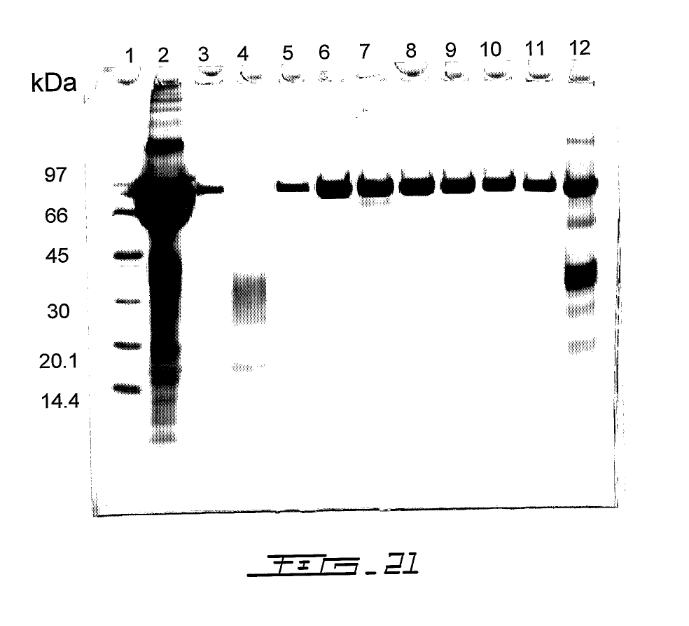

Stability of lactoferrin purified on Phenyl Sepharose™ HP resin in accordance with Example 14 was compared to DMV starting material. FIG. 22 clearly shows the stability at 4° C. of purified LF (lines 5, 7, 9, 10-12 vs. line 3 at time 0) from protein degradation over time as compared to DMV starting raw material (lines 4, 6 and 8 vs. line 2 at time 0) which showed extensive protein and fragment degradation.

FIG. 22 illustrates a silver stained SDS-PAGE gel electrophoresis of bovine lactoferrin (20 mg/ml), purified on Phenyl Sepharose™ HP resin by the surfactant-mediated HILIC method (Example 14) and subjected to different incubation times (0, 1, 2 and 5 days), in solution buffer (0.01M NaHCO₃, 0.001M citric acid, 0.01M NaCl, pH 7.2) at 4° C. Gel wells were loaded accordingly as follows:

| Product loaded | Lanes |
|---|---|
| LMW Pharmacia marker | 1 |
| T = 0 DMV lactoferrin starting material | 2 |
| T = 0 Phenyl Sepharose purified lactoferrin | 3 |
| T = 24 hrs at 4° C. DMV lactoferrin | 4 |
| T = 24 hrs at 4° C. Sepharose purified lactoferrin | 5 |
| T = 48 hrs at 4° C. DMV lactoferrin | 6 |
| T = 48 hrs at 4° C. Sepharose purified lactoferrin | 7 |
| T = 5 days at 4° C. DMV lactoferrin | 8 |
| T = 5 days at 4° C. Sepharose purified lactoferrin | 9 |
| T = 24 hrs at 4° C. Sepharose purified lactoferrin | 10 |
| T = 48 hrs at 4° C. Sepharose purified lactoferrin | 11 |
| T = 5 days at 4° C. Sepharose purified lactoferrin | 12 |

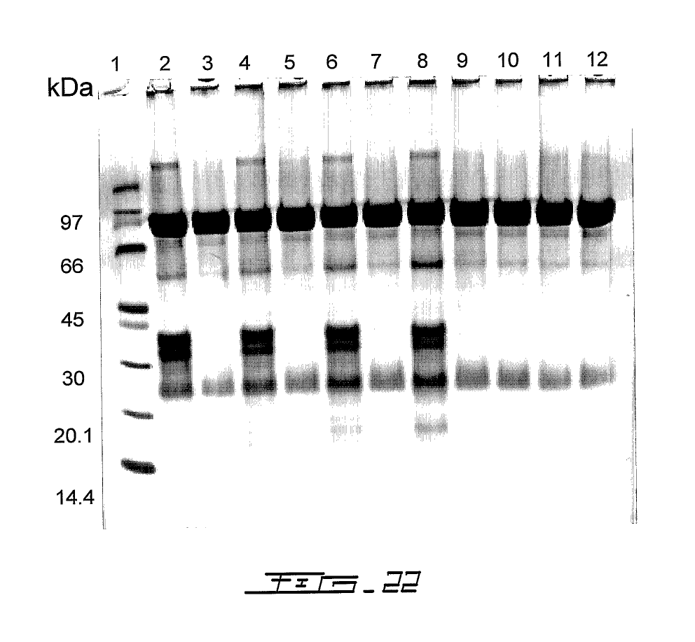

In addition, lactoferrin purified on Phenyl Sepharose™ HP resin in accordance with Example 14 was exposed to 30° C. incubation over 5 days. Again, purified lactoferrin clearly showed protein stability as evidenced by the absence of fragment appearance indicative of protein degradation (FIG. 23), which is also indicative at 30° C. of long term stability of purified lactoferrin over time, unlike DMV lactoferrin showing appearance of degradation fragments indicative of proteolysis even at 4° C. (FIG. 22).

FIG. 23 illustrates a silver stained SDS-PAGE gel electrophoresis of bovine lactoferrin (20 mg/ml), purified on Phenyl Sepharose™ HP resin, in accordance with Example 14 and subjected to different incubation times (0, 1, 2 and 5 days), in solution buffer (0.01M NaHCO₃, 0.001M citric acid, 0.01M NaCl, pH 7.2) at 30° C. Gel wells were loaded accordingly as follows:

| Product loaded | Lanes |
|---|---|
| LMW Pharmacia marker | 1 |
| DMV lactoferrin starting material prior to Phenyl Sepharose | 2 |
| purification (bef. 0.22 0.22 μm filtration) | |
| T = 0 day Phenyl Sepharose 3.4 L R3 purified lactoferrin | 3 |
| T = 0 day duplicate | 4 |
| T = 1 day Phenyl Sepharose 3.4 L R3 purified lactoferrin | 5 |
| T = 1 day duplicate | 6 |
| T = 2 days Phenyl Sepharose 3.4 L R3 purified lactoferrin | 7 |
| T = 2 days duplicate | 8 |
| T = 5 days Phenyl Sepharose 3.4 L R3 purified lactoferrin | 9 |
| T = 5 days duplicate | 10 |
| empty wells | 11 |
| empty wells | 12 |

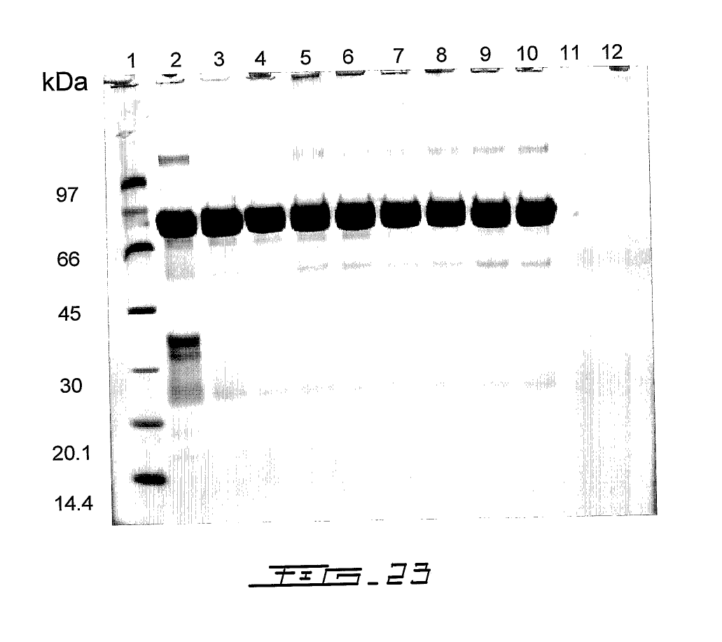

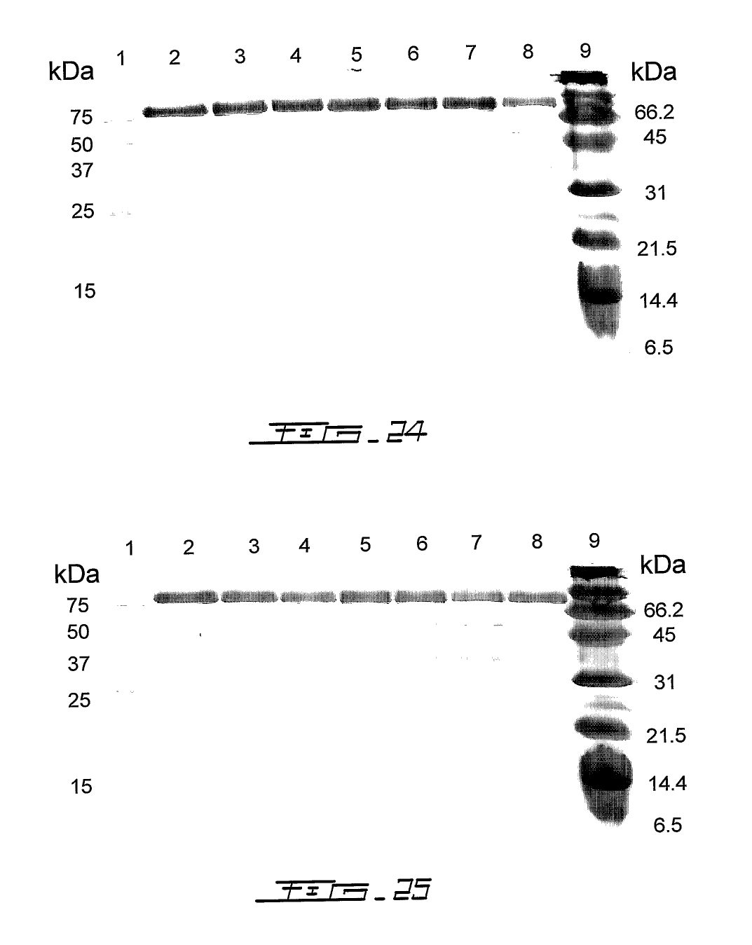

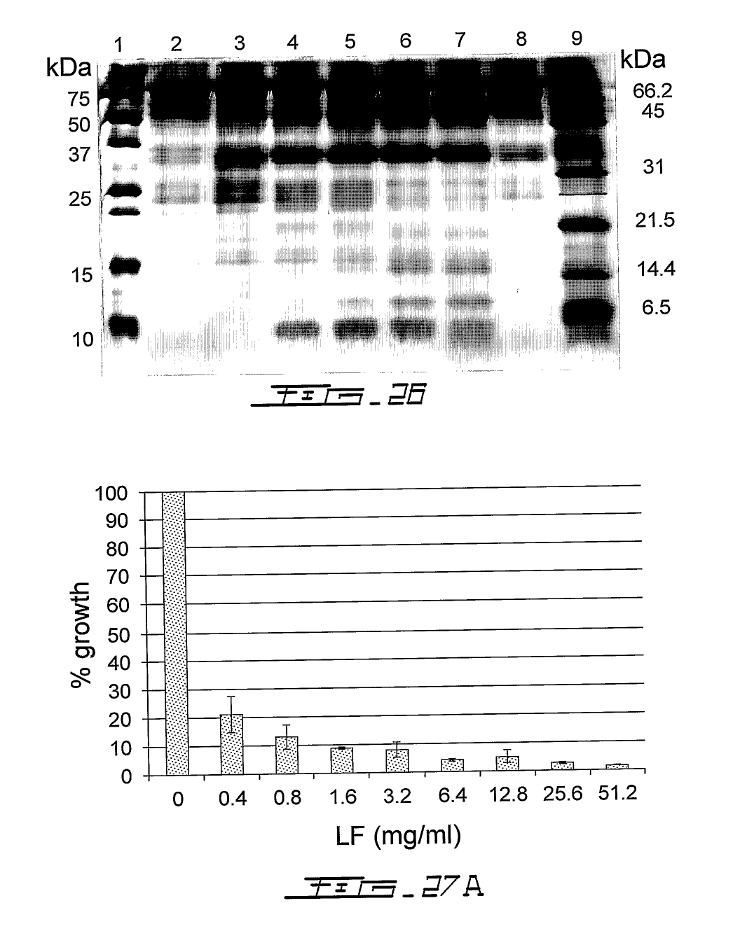

Furthermore, lactoferrin (106 mg per ml) purified on Phenyl Sepharose™ HP resin in accordance with Example 14 was incubated at room temperature in solution buffer (0.01M NaHCO₃, 0.001M citric acid, 0.01M NaCl, pH 7.2) over 6 months. FIG. 24 clearly shows remarkable protein stability of purified lactoferrin over time; this is strong evidence of complete enzyme contaminant removal responsible for non-purified lactoferrin degradation in solution. In fact, non-purified lactoferrin clearly shows evidence of protein and fragment degradation as evidenced by the appearance and increased intensity of smaller molecular weight fragments over time (FIG. 25). Extent of non-purified lactoferrin degradation is further demonstrated in FIG. 26 by the numerous silver stained fragments appearing over time unlike lactoferrin purified on Phenyl Sepharose™ HP in accordance with Example 14 showing remarkable stability over 6 months in solution (line 2 and 8, FIG. 26).

FIG. 24 illustrates a Coomassie blue stained SDS-PAGE gel electrophoresis of bovine lactoferrin (106 mg/ml) purified on Phenyl Sepharose™ HP resin in accordance with Example 14 and subjected to different incubation times (0, 14 days, 1 month, 3 months and 6 months) in solution buffer (0.01M NaHCO₃, 0.001M citric acid, 0.01M NaCl, pH 7.2) at room temperature. Gel wells were loaded accordingly as follows:

| Product loaded | Lanes |
|---|---|
| Molecular weight marker | 1 |
| T = 0 day DMV lactoferrin starting material prior to Phenyl | 2 |
| Sepharose purification (bef. 0.22 0.22 μm filtration) | |
| T = 0 day Phenyl Sepharose 3.4 L R5 purified lactoferrin | 3 |
| T = 14 days Phenyl Sepharose 3.4 L R5 purified lactoferrin | 4 |
| T = 1 month Phenyl Sepharose 3.4 L R5 purified lactoferrin | 5 |
| T = 3 months Phenyl Sepharose 3.4 L R5 purified lactoferrin | 6 |
| T = 6 months Phenyl Sepharose 3.4 L R5 purified lactoferrin | 7 |
| T = 6 months DMV lactoferrin starting material prior to Phenyl | 8 |
| Sepharose purification (bef. 0.22 0.22 μm filtration) | |
| Molecular weight marker | 9 |

FIG. 25 illustrates a Coomassie blue stained SDS-PAGE gel electrophoresis of non-purified bovine lactoferrin (107 mg/ml) subjected to different incubation times (0, 14 days, 1 month, 3 months and 6 months) in solution buffer (0.01M NaHCO₃, 0.001M citric acid, 0.01M NaCl, pH 7.2) at room temperature. Gel wells were loaded accordingly as follows:

| Product loaded | Lanes |
|---|---|
| Molecular weight marker | 1 |
| T = 0 day Phenyl Sepharose 3.4 L R5 purified lactoferrin | 2 |
| T = 0 day DMV lactoferrin starting material prior to Phenyl | 3 |
| Sepharose purification (bef. 0.22 0.22 μm filtration) | |
| T = 14 days DMV lactoferrin starting material prior to Phenyl | 4 |
| Sepharose purification (bef. 0.22 0.22 μm filtration) | |
| T = 1 month DMV lactoferrin starting material prior to Phenyl | 5 |
| Sepharose purification (bef. 0.22 0.22 μm filtration) | |
| T = 3 months DMV lactoferrin starting material prior to Phenyl | 6 |
| Sepharose purification (bef. 0.22 0.22 μm filtration) | |
| T = 6 months DMV lactoferrin starting material prior to Phenyl | 7 |
| Sepharose purification (bef. 0.22 0.22 μm filtration) | |
| T = 6 months Phenyl Sepharose 3.4 L R5 purified lactoferrin | 8 |
| Molecular weight marker | 9 |

FIG. 26 illustrates silver stained SDS-PAGE gel electrophoresis of non-purified bovine lactoferrin (107 mg/ml) subjected to different incubation times (0, 14 days, 1 month, 3 months and 6 months) in solution buffer (0.01M NaHCO₃, 0.001M citric acid, 0.01M NaCl, pH 7.2) at room temperature. Gel wells were loaded accordingly as follows:

| Product loaded | Lanes |
|---|---|
| Molecular weight marker | 1 |
| T = 0 day Phenyl Sepharose 3.4 L R5 purified lactoferrin | 2 |
| T = 0 day DMV lactoferrin starting material prior to Phenyl | 3 |
| Sepharose purification (bef. 0.22 0.22 μm filtration) | |
| T = 14 days DMV lactoferrin starting material prior to Phenyl | 4 |
| Sepharose purification (bef. 0.22 0.22 μm filtration) | |
| T = 1 month DMV lactoferrin starting material prior to Phenyl | 5 |
| Sepharose purification (bef. 0.22 0.22 μm filtration) | |
| T = 3 months DMV lactoferrin starting material prior to Phenyl | 6 |
| Sepharose purification (bef. 0.22 0.22 μm filtration) | |
| T = 6 months DMV lactoferrin starting material prior to Phenyl | 7 |
| Sepharose purification (bef. 0.22 0.22 μm filtration) | |
| T = 6 months Phenyl Sepharose 3.4 L R5 purified lactoferrin | 8 |
| Molecular weight marker | 9 |

### Example 18

### Lactoferrin Activity

Minimal inhibitory concentrations (MIC) were determined in triplicate from three separate experiments (n=9 per value) by broth microdilution techniques according to the National Committee for Clinical Laboratory Standards (National Committee for Clinical Laboratory Standards. 1999. Performance standards for susceptibility tests for bacteria isolated from animals; Approved Standard M31-A. National Committee for Clinical Laboratory Standards, Villanova, Pa.). Serial 2-fold dilutions of antibacterial agents were inoculated with an overnight culture at a final inoculum's concentration of 5.6×10⁵ colony forming units per ml.

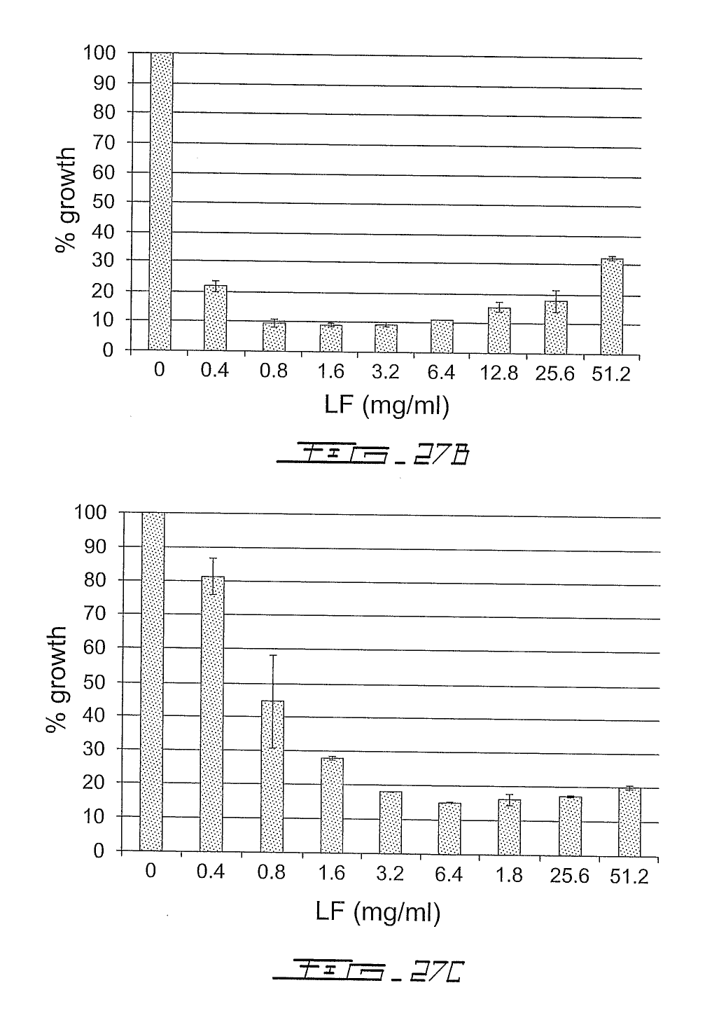

Compared to the lactoferrin purified on Phenyl Sepharose™ HP resin in accordance with Example 14, commercial non-purified lactoferrin from the same supplier as well as other suppliers available on the market did not display the same activity (FIG. 27). Clearly, the lactoferrin purified in accordance with Example 14 was more potent and did not lose its activity at higher concentration of lactoferrin in the medium. MIC of lactoferrin was estimated at 51.2 mg per ml for S. aureus strain SHY-97-4320. None of the non-purified lactoferrin preparations available on the market were able to display a MIC. Similar results were obtained with another S. aureus strain ATTC 29213.

The lactoferrin purified according to the process described in this invention is more potent. Indeed, purified lactoferrin from DMV at 6.4 mg per ml inhibited in 24 h the growth of S. aureus by 96% (FIG. 27a) compared to control growth (0 mg per ml). On the other hand, growth inhibition of commercial non-purified lactoferrin (LFnp) from DMV was 89% (FIG. 27b) while effect of LFnp from Morinaga was 84% (FIG. 27c), from Euro was 78% (FIG. 27d) and LFnp from Glanbia was only 69% (FIG. 27e). At concentration greater than 6.4 mg per ml, these differences in favor of the purified lactoferrin (LFp) according to process described in this invention were even more pronounced.

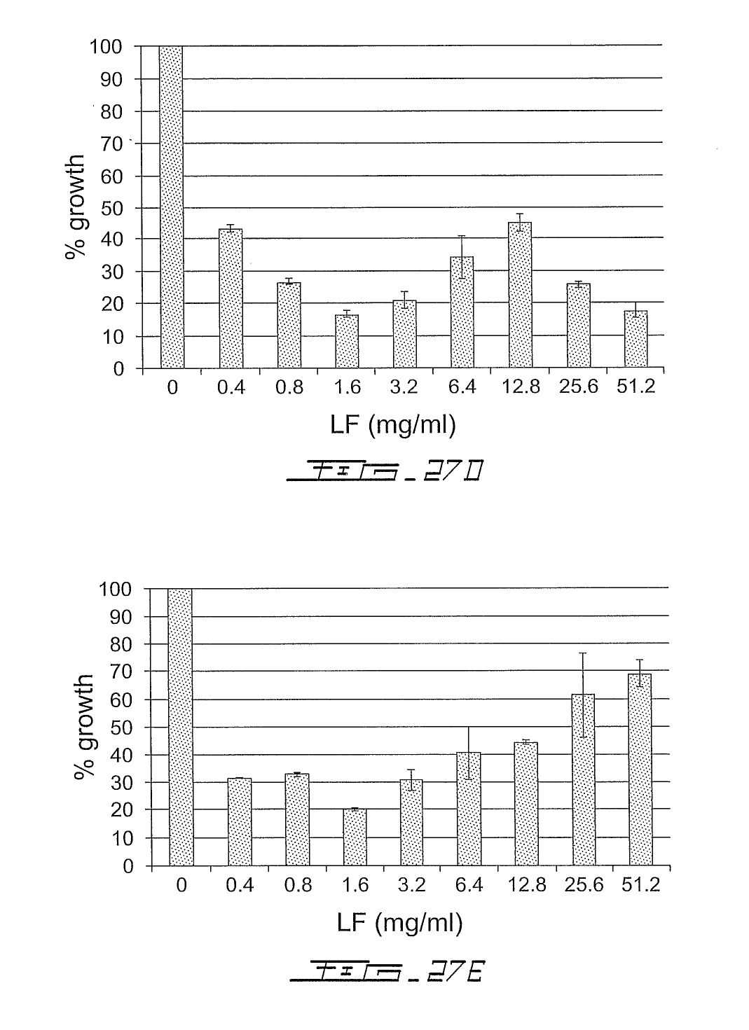

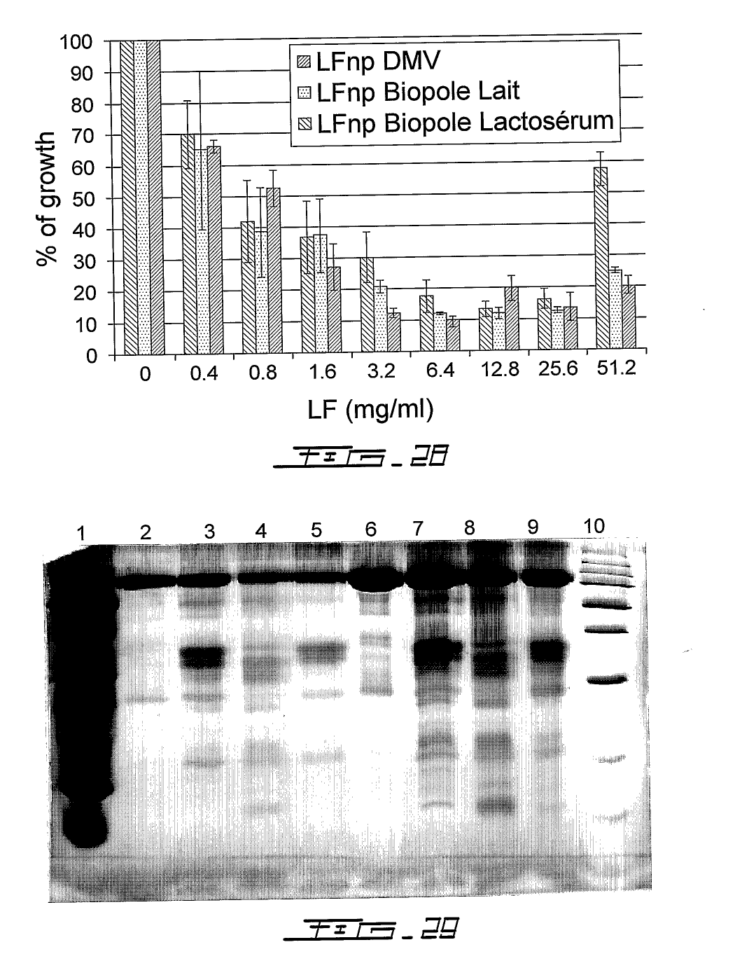

Source of starting material from different commercial supplier was unknown (i.e., extracted from milk or from lactoserum). However, one supplier provided us with lactoferrin extracted from milk rather than lactoserum. As it can be seen in FIG. 28, growth inhibitory activity of commercial non-purified lactoferrin (LFnp) was equivalent between DMV lactoferrin and lactoferrin extracted from milk by Biopole. However, LF extracted by Biopole from lactoserum clearly lost its growth inhibitory activity at high concentration. This greater re-bounding effect of lactoferrin extracted from lactoserum is indicative, among other things, of either greater lactoferrin degradation or greater amount of minor contaminants or degradation products (see FIG. 29) interfering with the activity of lactoferrin on bacterial growth. Similar results were obtained with another S. aureus strain SHY97-4320.

FIG. 29 illustrates silver stained SDS-PAGE gel electrophoresis of bovine lactoferrin from DMV purified according to Example 14 (106 mg/ml), non-purified bovine lactoferrin from DMV (107 mg/ml), non-purified bovine lactoferrin extracted from milk by Biopole (110 mg/ml) and non-purified bovine lactoferrin extracted from lactoserum by Biopole (124 mg/ml). Gel wells were loaded accordingly as follows:

| Product loaded | Lanes |
|---|---|
| Molecular weight marker | 1 |
| 2 μg of DMV lactoferrin purified on Phenyl Sepharose 3.4 L R5 | 2 |
| 2 μg of non-purified DMV lactoferrin | 3 |
| 2 μg of Biopole lactoferrin extracted from lactoserum according to | 4 |
| manufacturer | |
| 2 μg of Biopole lactoferrin extracted from milk according to | 5 |
| manufacturer | |
| 6.5 μg of DMV lactoferrin purified on Phenyl Sepharose 3.4 L R5 | 6 |
| 6.5 μg of non-purified DMV lactoferrin | 7 |
| 6.5 μg of Biopole lactoferrin extracted from lactoserum according | 8 |
| to manufacturer | |
| 6.5 μg of Biopole lactoferrin extracted from milk according to | 9 |
| manufacturer | |
| Molecular weight marker | 10 |

Furthermore, it can be seen in the following Example that the in vivo response of the purified lactoferrin versus the commercial non-purified lactoferrin from the same supplier at the same dose was again superior in favor of the purified form according to the process described in this invention.

### Example 19

### Lactoferrin Activity and Immune Response

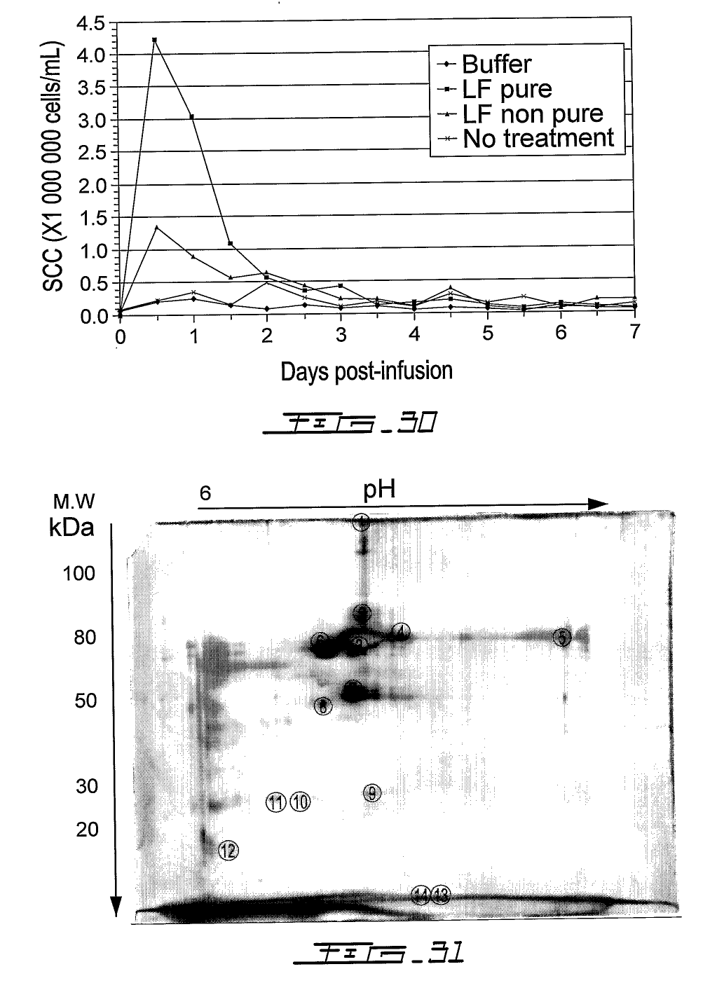

Infusion of lactoferrin into the mammary gland of lactating cows stimulates the migration of polymorphonuclear neutrophils (PMN) into the mammary gland of cows to fight infections. To test in vivo the biological activity of purified lactoferrin according to the process described in this invention vs commercially available lactoferrin, 6 lactating cows were randomly infused into one quarter of their mammary gland with one the following treatments: no infusion treatment, citrate buffer (0.001 citric acid; 0.01 NaHCO₃; 0.1M NaCl; pH 7.2) infusion, infusion of 1 g of purified LF according to Example 14 or infusion of 1 g of commercial non-purified LF. Results showed that purified LF had a greater biological activity as demonstrated by the larger migration of PMN as measured by somatic cell count (SCC) response as compared to non-purified lactoferrin (FIG. 30).

Again, the lactoferrin purified in accordance to the process described in this invention was more potent than non-purified lactoferrin (commercially available).

### Example 20

### Proteomic Analysis of Purified Lactoferrin Preparation

To identify proteins/peptides present in purified lactoferrin preparation, a classical approach was used consisted of 2D-PAGE, followed by tryptic digestion of the protein/peptide spots and LC-MS/MS analysis of the tryptic peptides.

For 2D-PAGE separation of purified lactoferrin (FIG. 31), 2 μl (10.6 mg/ml) was mixed with 125 μl of the rehydration buffer composed of 8 M urea, 0.5% (w/v) CHAPS, 2% (v/v) IPG Buffer pH 6-11, 0.002% bromophenol blue and 20 mM DTT. Sample was loaded on 7 cm IEF Buffer Strip pH 6-11 and proteins were resolved using IPGphor system (Pharmacia Biotech) according to manufacturer's recommendation. Following the IEF separation of proteins, Buffer Strips were equilibrated for 15 min in Equilibration Buffer composed of 6M urea in 50 mM Tris-HCl pH 8.8, 30% glycerol, 2% SDS, 1% DTT and 0.002% bromophenol blue. In order to alkylate proteins, Buffer Strip was transferred to Equilibration Buffer where 1% DTT was replaced with 2.5% iodoacetamide. After 15 min incubation, Buffer Strip was placed over 10% acrylamide gel (Laemmli method) and covered with 2% agarose in running buffer.

In the second dimension, proteins were resolved for approx. 1.5 hrs. at 120V. To detect proteins, gel was stained with non-reducing silver staining method and image of the gel was recorded with the CD camera.

Most of the protein spots concentrated around 70 kDa, pl 8.0 region, i.e., expected MW and pl values for bovine lactoferrin. These and the other protein spots (as indicated by numbers on the gel image) were cut-out from the gel and subjected to digestion with a sequence grade trypsin (Promega). Resulted peptide fragments were desalted by reverse-phase adsorption/wash/desorption using C18 silica (ZipTip, Millipore).

Peptides were dissolved in 0.2% formic acid and analyzed by LC-MS/MS using ESI-QTOF Global (Micromass). Direct Data Acquisition (DDA) experiment was performed with glu-fibrinogen as the Lock Mass calibration standard. Raw sequence data files (PKL files) were analyzed and the results scored using Mascot Search algorithm (http://www.matrixscience.com/search_form_select.html).

Spots #1-6 were identified as full-length lactoferrins. Difference in IP and/or M.W. is probably caused by minor truncations of the protein and/or post-translational modification. Spot #7 is clearly C-terminal part of lactoferrin, while spots 12-14 are derived from the N-terminus of the protein.

It is difficult to identify both low and high abundance proteins. For this reason, spot #9, which was very low, was not identified, and scores for the spots #10 and #11 are too low for clear identification. However, spots 8 and 13, even though very low, were similar to keratin 4 isoform 2 [Bos taurus].

In conclusion, lactoferrin purified in accordance to the process described in this invention is substantially pure, being free of degrading enzyme.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. (canceled)

2. A method for purifying lactoferrin comprising the steps of:
contacting in a bind-and-elute mode and in an adsorptive fashion a solution of lactoferrin, with a hydrophilic adsorbent in the presence of an excluded solute;
applying a decreasing concentration gradient of the excluded solute; and
collecting a fraction containing lactoferrin substantially free of contaminant enzyme and/or lactoferrin inhibitor.

3. (canceled)

4. A method for purifying lactoferrin comprising the steps of:
contacting in a bind-and-elute mode and in an adsorptive fashion a solution of lactoferrin, with a hydrophobic adsorbent in the presence of a surfactant, and in the presence of an excluded solute;
applying a decreasing concentration gradient of the excluded solute; and
collecting a fraction containing lactoferrin substantially free of contaminant enzyme and/or lactoferrin inhibitor.

5. The method as defined in claim 2, wherein the excluded solute is selected from the group consisting of ammonium sulfate, sodium sulfate, potassium phosphate and sodium chloride.

6-8. (canceled)

9. The method as defined in claim 2, wherein the surface of the hydrophilic adsorbent is substantially composed of silica, calcium silicate, hydroxyapatite, titanium dioxide or zirconia.

10-11. (canceled)

12. The method as defined in claim 4, wherein the surface of the hydrophobic adsorbent is substantially composed of polystyrene/divinyl benzene co-polymer.

13-15. (canceled)

16. The method as defined in claim 2, wherein the hydrophilic adsorbent is Superdex, Sephadex, Superose, Sephacryl, Sepharose, cross-linked agarose, a TOYOPEARL® size exclusion protein chromatography medium, TOYOPEARL® Ether or FRACTOGEL® EMD BioSEC.

17. The method as defined in claim 4, wherein the hydrophobic adsorbent is Phenyl Sepharose.

18-20. (canceled)

21. A method for purifying lactoferrin comprising the steps of:
adsorbing a solution of lactoferrin to a hydrophobic adsorbent in the presence of an aqueous acidic solution containing concentration of a charged excluded solute;
applying an increasing pH gradient; and
collecting a fraction containing lactoferrin substantially free of contaminant enzyme and/or lactoferrin inhibitor by applying a decreasing concentration gradient of the excluded solute.

22. The method as defined in claim 21, wherein the excluded solute is selected from the group consisting of ammonium sulfate, sodium sulfate, potassium phosphate and sodium chloride.

23. The method as defined in claim 21, wherein the adsorbent is a phenyl-functional hydrophobic interaction chromatography medium selected from the group consisting of Phenyl Sepharose HP, Phenyl Sepharose Fast Flow (low substitution), Phenyl Sepharose Fast Flow (high substitution), TOYOPEARL® Phenyl-650, TSKgel Phenyl-5PW, FRACTOGEL® EMD Phenyl 650 and Poros HP2.

24-25. (canceled)

26. A method as defined in claim 21, wherein the pH of the aqueous acidic solution is between 3.0 and 4.5.

27-31. (canceled)

32. A lactoferrin substantially free of contaminant enzyme and/or lactoferrin inhibitor.

33. (canceled)

34. A lactoferrin comprising at least one enzyme inhibitor preventing degradation of lactoferrin.

35-37. (canceled)

38. The lactoferrin as defined in claim 34, having a minimal inhibitory concentration of at least 1 mg/ml.

39-42. (canceled)

43. The method as defined in claim 4, wherein the excluded solute is selected from the group consisting of ammonium sulfate, sodium sulfate, potassium phosphate and sodium chloride.
